(19) United States
(12) Patent Application Publication
(10) Pub. No.: US 2003/0059889 A1
(43) Pub. Date: Mar. 27, 2003

(54) Tumor necrosis factor receptor related protein-1 gene and protein

(76) Inventors: Dale Lesley Bodian, Whippany, NJ (US); Mark Aron Labow, Westfield, NJ (US); Craig Stephen Mickanin, Basking Ridge, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/231,426

(22) Filed: Aug. 28, 2002

Related U.S. Application Data
(60) Provisional application No. 60/317,151, filed on Sep. 5, 2001.

(51) Int. Cl. C12Q 1/68; C07H 21/04; C07K 14/715; C12P 21/02; C12N 5/06

(52) U.S. Cl. 435/69.1; 435/320.1; 435/325; 530/350; 536/23.5; 435/6

(57) ABSTRACT

Disclosed is a tumor necrosis factor receptor related protein 1 gene and gene product. In particular, the invention relates to a protein that is homologous to known tumor necrosis factor receptors, nucleic acid molecules that encode such a protein, antibodies that recognize the protein, methods for diagnosing and treating disorders, such as inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders, methods of identifying molecules that bind and/or modulate the activity of TRP-1 protein, and methods of identifying molecules that bind to a nucleic acid encoding TRP-1 protein and/or modulate the transcription or translation of the nucleic acid encoding TRP-1 protein.

### CROSS-REFERENCE TO RELATED APPLICATIONS

This application claims the benefit of U.S. provisional application No. 60/317,151 filed Sep. 5, 2001. The aforementioned application is incorporated herein by reference.

### BACKGROUND OF INVENTION

1. Field of the Invention

This invention relates to tumor necrosis factor receptors. In particular, the invention relates to a novel protein that is homologous to known tumor necrosis factor receptors, nucleic acid molecules that encode such a protein, antibodies that recognize the protein, methods for diagnosing and treating disorders, such as inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders, and methods for identifying molecules that bind the protein.

2. Description of the Related Art

Members of the tumor necrosis factor receptor superfamily (TNFRSF) are cell surface proteins expressed on many types of human cells which are involved in regulating a wide variety of biological activities such as inflammation, immunity, cell proliferation, differentiation, viral replication, neurodegeneration, bone growth, hematopoiesis and programmed cell death or apoptosis. These receptor proteins transduce extracellular signals into cells through interaction with ligands known as cytokines which generally belong to the tumor necrosis factor family.

Representative proteins of the TNFRSF possess the typical structure of cell surface receptors including extracellular, transmembrane and cytoplasmic domains. TNFRSF members share similar multiple cysteine-rich domains, i.e., pseudorepeats, within the extracellular domain, which contribute to ligand binding. In general, TNFRSF members contain a variable number of the pseudorepeats, each of which possesses approximately six cysteine residues distributed within a stretch of about 40 amino acids.

TNFRSF members can be classified into two main subgroups, that either possess or lack a conserved region of about 80 amino acids known as the “death domain” localized to the receptor's cytoplasmic domain, which is responsible for mediating cell death as described, e.g., in Tartaglia et al., Cell, Vol. 74, pp. 845-853 (1993). The cytoplasmic domains of TNFRSF members are of variable length and, other than in the death domain, are unrelated in sequence, which is suggestive of different modes of signalling. The death domain couples each receptor to caspase cascades that cause apoptosis or to kinase cascades that initiate gene expression through two transcription factors known as NF-KB (nuclear factor-KB) and AP-1 (activator protein 1).

At the present time over 20 different members of the TNFRSF have been identified, particularly with the aid of massive DNA sequencing and bioinformatic studies. Biochemical and genetic studies of the TNFRSF members have provided insights into the cellular signalling mechanisms by which the TNFRSF regulates various biological processes such as apoptosis. For example, Eby et al., J. Biol. Chem., Vol. 275, No. 19, pp. 15336-15342 (2000), describe the isolation and characterization of a novel member of the TNFRSF designated TAJ (for Toxicity and c-Jun N-terminal kinase inducer) which is expressed during embryonic development and which activates the c-Jun N-terminal kinase pathway. Although TAJ lacks a death domain, it induces apoptosis by a caspase-independent mechanism in cells transfected with an expression vector encoding TAJ.

TNFRSF proteins have important therapeutic applications. Enbrel, a soluble form of TNFR-1 has been used in the treatment of arthritis as described, e.g., in Pisetsky, NEJM, Vol. 342, p. 810 (2000). Remicade, a chimeric antibody, that inhibits binding between TNFR-1 and its ligand has also be used in the treatment of arthritis as described, e.g., in Pisetsky, supra. A soluble form of the TNFRSF ligand, TRAIL, is in preclinical trials for the treatment of cancer as described, e.g., in Ashkenazi et al., J. Clin. Invest, Vol. 104, pp. 155162 (1999). Other products, including soluble forms of both TNFRSF proteins and their ligands (e.g., RANK, osteoprotegerin, 4-1BB and CD40L) are at various stages of development as potential therapeutic agents for a variety of immune disorders, inflammation, osteoporosis and cancer.

In view of the pleiotropic role of TNFRSF members in regulating various biological activities, the identification of new members of TNFRSF would allow the development of additional diagnostic and therapeutic candidates to diagnose and treat various disorders, such as inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders. In addition, the identification of new TNFRSF members would permit the screening of various drugs to identify those compounds suitable for further, in-depth studies of therapeutic applications. The identification of new TNFRSF members would also enable the identification of ligands which bind TNFRSF proteins, which in turn would enable development of various therapeutics, e.g., small molecules and monoclonal antibodies, to counteract or enhance the biological effect of such ligands, and the elucidation of cellular signalling mechanisms for new TNFRSF members.

### SUMMARY OF THE INVENTION

The present invention relates to tumor necrosis factor receptors, in particular to a splice variant of human X-linked ectodysplasin-A2 receptor (XEDAR) referred herein as tumor necrosis factor receptor related protein-1 (TRP-1), and nucleotide sequences encoding TRP-1.

In one aspect, the invention provides an isolated polypeptide comprising an amino acid sequence as set forth in SEQ ID NO: 1. Furthermore, the invention provides an isolated polypeptide consisting of an amino acid sequence as set forth in SEQ ID NO: 1. For convenience, the polypeptide consisting of the amino acid sequence as set forth in SEQ ID NO: 1 will be designated as TRP-1. Such a polypeptide, fragment thereof or variant of the polypeptide, is expressed in various tissues, predominantly in testes, uterus, prostate, mammary gland and stomach. Fragments of the isolated polypeptide having an amino acid sequence as set forth in SEQ ID NO: 1 will comprise polypeptides comprising from about 5-269 amino acids, preferably from about 10 to about 161 amino acids, more preferably from about 20 to about 136 amino acids, and most preferably from about 41 to about 119 amino acids. Such fragments also form a part of the present invention and have different uses as described below. In accordance with this aspect of the invention a novel polypeptide of human origin is provided as well as biologically, diagnostically or therapeutically useful fragments, variants and derivatives of the polypeptide, and variants and derivatives of the fragments.

In another aspect, the invention provides an isolated DNA comprising a nucleotide sequence that encodes a polypeptide as mentioned above. In particular, the invention provides:

(a) a nucleotide sequence that encodes a protein comprising the amino acid sequence set forth in SEQ ID NO: 1;

(b) a nucleotide sequence encoding amino acid residues 1-136 of SEQ ID NO: 1;

(c) a nucleotide sequence as set forth in nucleotides 113-922 of SEQ ID NO: 2;

(d) a nucleotide sequence comprising the nucleotide sequence set forth in SEQ ID NO: 3;

(e) a nucleotide sequence capable of hybridizing under high stringency conditions to the nucleotide sequence set forth in nucleotides 113-922 SEQ ID NO: 2; and

(f) a nucleotide sequence which is complementary to the nucleotide sequence of (a), (b), (c), (d) or (e).

Also provided are nucleic acid sequences comprising at least about 15 bases, preferably at least about 20 bases, more preferably a nucleic acid sequence comprising about 30 contiguous bases of SEQ ID NO: 2. Also provided are isolated nucleic acid sequences, comprising a coding region that encodes a splice variant of TRP-1 polypeptide, wherein the TRP-1 polypeptide is encoded by a nucleotide sequence as set forth in SEQ ID NO: 3. Also within the scope of the present invention are nucleic acids that are substantially similar to the nucleic acid with the nucleotide sequence as set forth in SEQ ID NO: 2 or SEQ ID NO: 3. In a preferred embodiment, the isolated DNA takes the form of a vector molecule comprising at least a fragment of a DNA of the present invention, in particular comprising the DNA consisting of a nucleotide sequence as set forth in nucleotides 113-922 of SEQ ID NO: 2 or a nucleotide sequence of SEQ ID NO: 3.

Another aspect of the present invention encompasses a method for the diagnosis of disorders selected from the group consisting of inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders in a human which includes detecting elevated or reduced transcription of messenger RNA (mRNA) transcribed from the natural endogenous human gene encoding the novel TRP-1 polypeptide in an appropriate tissue or cell from a human, where such elevated or reduced transcription is diagnostic of the human's affliction with such a disorder. In particular, the natural endogenous human gene encoding the novel polypeptide consists of the amino acid sequence set forth in SEQ ID NO: 1 comprising the genomic nucleotide sequence set forth in SEQ ID NO: 3. In one embodiment of the present invention, the diagnostic method comprises contacting a sample of the appropriate tissue or cell or contacting an isolated RNA or DNA molecule derived from that tissue or cell with an isolated nucleotide sequence of at least about 15-20 nucleotides in length that hybridizes under high stringency conditions with the isolated nucleotide sequence encoding the novel polypeptide having an amino acid sequence set forth in SEQ ID NO: 1. Another embodiment of the assay aspect of the invention provides a method for the diagnosis of disorders selected from the group consisting of inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders in a human which requires detecting the amount of the TRP-1 polypeptide or fragments thereof in a certain tissue or cell from a human suffering from such a disorder, where the presence of an elevated or reduced amount of the polypeptide or fragments thereof, relative to the amount of the polypeptide or fragments thereof in the respective tissue or cell of a healthy individual, is diagnostic of the human's suffering from such a disorder.

In accordance with one embodiment of this aspect of the invention there are provided anti-sense polynucleotides that can regulate transcription of the gene encoding the novel TRP-1 polypeptide; in another embodiment, double stranded RNA is provided that can regulate expression of the novel TRP-1 polypeptide.

Another aspect of the invention provides a process for producing the aforementioned polypeptides, polypeptide fragments, variants and derivatives of the polypeptides, and variants and derivatives of the polypeptide fragments. In a preferred embodiment of this aspect of the invention there are provided methods for producing the aforementioned TRP-1 polypeptide comprising culturing host cells having incorporated therein an expression vector containing an exogenously-derived nucleotide sequence encoding such a polynucleotide under conditions sufficient for expression of the polypeptide in the host cell, thereby causing expression of the polypeptide, and optionally recovering the expressed polypeptide. In a preferred embodiment of this aspect of the present invention, there is provided a method for producing a polypeptide comprising or consisting of an amino acid sequence as set forth in SEQ ID NO: 1 or a fragment thereof, which comprises culturing a host cell having incorporated therein an expression vector containing an exogenously-derived polynucleotide encoding a polypeptide comprising or consisting of an amino acid sequence as set forth in SEQ ID NO: 1, under conditions sufficient for expression of such a polypeptide in the host cell, thereby causing the production of an expressed polypeptide, and optionally recovering the expressed polypeptide. Preferably, in any of such methods the exogenously derived polynucleotide comprises or consists of the nucleotide sequence set forth in nucleotides 113-922 of SEQ ID NO: 2, or the nucleotide sequence set forth in SEQ ID NO: 3. In accordance with another aspect of the invention there are provided products, compositions, processes and methods that utilize the aforementioned polypeptides and polynucleotides for, inter alia, research, biological, clinical and therapeutic purposes.

In certain additional preferred embodiments of this aspect of the invention there is provided an antibody or a fragment thereof which specifically binds to a polypeptide that comprises the amino acid sequence set forth in SEQ ID NO: 1, i.e., TRP-1. In certain particularly preferred embodiments in this regard, the antibodies are highly selective for human TRP-1 polypeptides or portions of human TRP-1 polypeptides.

In a further aspect, an antibody or fragment thereof is provided that binds to a fragment or portion of the amino acid sequence set forth in SEQ ID NO: 1.

In another aspect, methods of treating a disorder in a subject, where the disorder is mediated by or associated with an increase or decrease in TRP-1 gene expression or an increase or decrease in the presence of TRP-1 polypeptide in a certain tissue or cell by the administration of an effective amount of an antibody that binds to a polypeptide with the amino acid sequence set out in SEQ ID NO: 1, or a fragment or portion thereof to the subject are provided. Also provided are methods for the diagnosis of a disorder associated with an increase or decrease in TRP-1 gene expression or an increase or decrease in the presence of the TRP-1 polypeptide in a subject, which comprises utilizing an antibody that binds to a polypeptide with the amino acid sequence set out in SEQ ID NO: 1, or a fragment or portion thereof, in an immunoassay.

In another aspect, a method of suppressing inflammation in a subject is provided which comprises administering a therapeutically effective amount of a soluble TRP-1 polypeptide, wherein the TRP-1 polypeptide has an amino acid sequence as set forth in amino acids 1-136 of SEQ ID NO: 1 or a significant sub-fragment from this region including no less then 10 continuous amino acids derived from amino acids 1-136.

In yet another aspect, the invention provides host cells which can be propagated in vitro, preferably vertebrate cells, in particular mammalian cells, or bacterial cells, which are capable upon growth in culture of producing a polypeptide that comprises the amino acid sequence set forth in SEQ ID NO: 1 or fragments thereof, where the cells contain transcriptional control DNA sequences, preferably other than human TRP-1 transcriptional control sequences, where the transcriptional control sequences control transcription of DNA encoding a polypeptide with the amino acid sequence according to SEQ ID NO: 1 or fragments thereof.

In yet another aspect of the present invention there are provided assay methods and kits comprising the components necessary to detect above-normal or below-normal expression of polynucleotides encoding a polypeptide comprising an amino acid sequence as set forth in SEQ ID NO: 1, or polypeptides comprising an amino acid sequence set forth in SEQ ID NO: 1, or fragments thereof, in body tissue samples derived from a patient, such kits comprising, e.g., antibodies that bind to a polypeptide comprising an amino acid sequence set forth in SEQ ID NO: 1, or to fragments thereof, or oligonucleotide probes that hybridize with polynucleotides of the invention. In a preferred embodiment, such kits also comprise instructions detailing the procedures by which the kit components are to be used.

In another aspect, the invention is directed to use of a polypeptide comprising an amino acid sequence set forth in SEQ ID NO: 1 or fragment thereof, polynucleotide encoding such a polypeptide or a fragment thereof, or antibody that binds to the polypeptide comprising an amino acid sequence set forth in SEQ ID NO: 1 or a fragment thereof in the manufacture of a medicament to treat disorders selected from the group consisting of inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders.

Another aspect is directed to pharmaceutical compositions comprising a polypeptide comprising or consisting of an amino acid sequence set forth in SEQ ID NO: 1 or fragment thereof, a polynucleotide encoding such a polypeptide or a fragment thereof, or antibody that binds to such a polypeptide or a fragment thereof, in conjunction with a suitable pharmaceutical carrier, excipient or diluent, for the treatment of the aforementioned disorders.

In another aspect, the invention is directed to methods for the identification of molecules that can bind to a polypeptide comprising or consisting of an amino acid sequence set forth in SEQ ID NO: 1 and/or modulate the activity of a polypeptide comprising or consisting of an amino acid sequence set forth in SEQ ID NO: 1 or molecules that can bind to nucleic acid sequences that modulate the transcription or translation of a polynucleotide encoding a polypeptide comprising or consisting of an amino acid sequence set forth in SEQ ID NO: 1. Such methods include, but are not limited to, those disclosed in, e.g., U.S. Pat. Nos. 5,541,070; 5,567,317; 5,593,853; 5,670,326; 5,679,582; 5,856,083; 5,858,657; 5,866,341; 5,876,946; 5,989,814; 6,010,861; 6,020,141; 6,030,779; and 6,043,024. Molecules identified by such methods also fall within the scope of the present invention.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

FIG. 1 depicts the alignment of the peptide sequences of TRP-1 and XEDAR. The alignment was generated with ClustalW then manually adjusted to match the exon boundaries. The sequences differ by a K/R substitution in the second cysteine-rich pseudorepeat and an apparently alternatively spliced exon in the cytoplasmic region. The predicted transmembrane region is underlined.

FIG. 2 depicts the alignment of the cysteine-rich domains of TRP-1 and other TNFRSF members. Unaligned residues are displayed in lowercase. The six conserved cysteines and the conserved Y are highlighted.

FIG. 3 depicts the alignment of TRP-1 and human TNFRSF19. The alignment was generated automatically by CLUSTAL W (see Thompson et al., Nucl. Acids Res., Vol. 22, pp. 4673-4680 (1994)). *Represents residues identical between the two sequences; and and : mark conserved residues.

### DETAILED DESCRIPTION OF THE INVENTION

All patent applications, patents and literature references cited herein are hereby incorporated by reference in their entirety.

In practicing the present invention, many conventional techniques in molecular biology, microbiology and recombinant DNA are used. These techniques are well known and are explained in, for example, Current Protocols in Molecular Biology, F. M. Ausebel, ed., Vols. I, II and III (1997); Sambrook et al., Molecular Cloning: A Laboratory Manual, 2^{nd} Edition, Cold Spring Harbor Laboratory Press, Cold Spring Harbor, N.Y. (1989); DNA Cloning: A Practical Approach, D. N. Glover, ed., Vols. I and II (1985); Oligonucleotide Synthesis, M. L. Gait, ed. (1984); Nucleic Acid Hybridization, Hames and Higgins, eds. (1985); Transcription and Translation, Hames and Higgins, eds. (1984); Animal Cell Culture, R. I. Freshney, ed. (1986); Immobilized Cells and Enzymes, IRL Press (1986); Perbal, “A Practical Guide to Molecular Cloning”; the series, Methods in Enzymology, Academic Press, Inc. (1984); Gene Transfer Vectors for Mammalian Cells, J. H. Miller and M. P. Calos, eds., Cold Spring Harbor Laboratory (1987); and Methods in Enzymology, Wu and Grossman and Wu, eds., respectively, Vols. 154 and 155.

The term “nucleotide sequence” is used interchangeably herein with the terms “polynucleotide” and “nucleic acid.”

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g., where only changes in amino acids not affecting the polypeptide function occur. Desirably the substantially similar nucleotide sequence encodes the polypeptide encoded by the reference nucleotide sequence. The percentage of identity between the substantially similar nucleotide sequence and the reference nucleotide sequence desirably is at least 80%, more desirably at least 85%, preferably at least 90%, more preferably at least 95%, still more preferably at least 99%. Sequence comparisons are carried out using a Smith-Waterman sequence alignment algorithm (see, e.g., Waterman, “M.S. Introduction to Computational Biology: Maps, Sequences and Genomes”, Chapman & Hall, London (1995) ISBN 0-412-99391-0, or at http://www-hto.usc.edu/software/seqaln/index.html). The locals program, version 1.16, is used with following parameters: match: 1, mismatch penalty: 0.33, open-gap penalty: 2, extended-gap penalty: 2.

A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% SDS, 0.5 M NaPO₄, 1 mM EDTA at 50° C. with washing in 1×SSC, 0.1% SDS at 50° C., more desirably still in 7% SDS, 0.5 M NaPO₄, 1 mM EDTA at 50° C. with washing in 0.5×SSC, 0.1% SDS at 50° C., preferably in 7% SDS, 0.5 M NaPO₄, 1 mM EDTA at 50° C. with washing in 0.1×SSC, 0.1% SDS at 50° C., more preferably in 7% SDS, 0.5 M NaPO₄, 1 mM EDTA at 50° C. with washing in 0.1×SSC, 0.1% SDS at 65° C., yet still encodes a functionally equivalent gene product.

“Elevated transcription of mRNA” refers to a greater amount of mRNA transcribed from the natural endogenous human gene encoding the novel polypeptide of the present invention present in an appropriate tissue or cell of an individual suffering from a disorder including, but not limited to, inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders, in particular at least about twice, preferably at least about five times, more preferably at least about 10 times, most preferably at least about 100 times the amount of mRNA found in corresponding tissues in humans who do not suffer from such a disorder. Such elevated level of mRNA may eventually lead to increased levels of protein translated from such mRNA in an individual suffering from such a disorder as compared with a healthy individual.

“Reduced transcription of mRNA” refers to a lower amount of mRNA transcribed from the natural endogenous human gene encoding the novel polypeptide of the present invention present in an appropriate tissue or cell of an individual suffering from a disorder including, but not limited to, inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders, in particular at least about two times, preferably at least about five times, more preferably at least about 10 times, most preferably at least about 100 times lower than the amount of mRNA found in corresponding tissues in humans who do not suffer from such a disorder. Such reduced level of mRNA may eventually lead to reduced levels of protein translated from such mRNA in an individual suffering from such a disorder as compared with a healthy individual.

A “host cell”, as used herein, refers to a prokaryotic or eukaryotic cell that contains heterologous DNA that has been introduced into the cell by any means, e.g., electroporation, calcium phosphate precipitation, microinjection, transformation, viral infection and the like.

“Heterologous” as used herein means “of different natural origin” or represent a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g., a different copy number or under the control of different regulatory elements.

A “vector” molecule is a nucleic acid molecule into which heterologous nucleic acid may be inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes”.

“Plasmids” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment, e.g., the natural environment if it is naturally-occurring. For example, a naturally-occurring polynucleotide, polypeptide or cell present in a living animal is not isolated, but the same polynucleotide, polypeptide or cell separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment. Such cells can include, e.g., cells obtained from an animal, plant, insect, microorganism, or a cell line, such as Chinese hamster ovary (CHO), NIH-3T3, HeLa and COS 7.

As used herein, the term “transcriptional control sequence” refers to DNA sequences, such as initiator sequences, enhancer sequences and promoter sequences, which induce, repress, or otherwise control the transcription of protein encoding nucleic acid sequences to which they are operably-linked.

As used herein, “human transcriptional control sequences” are any of those transcriptional control sequences normally found associated with the human gene encoding the novel TRP-1 polypeptide of the present invention as it is found in the respective human chromosome.

As used herein, “non-human transcriptional control sequence” is any transcriptional control sequence not found in the human genome.

The term “splice variant” means variant invention protein-encoding nucleic acids produced by differential processing of primary transcript(s) of genomic DNA, resulting in the production of more than one type of mRNA. cDNA derived from differentially processed transcript will encode the invention polypeptide that have regions of complete amino acid identity and regions having different amino acid sequences. Accordingly, the same genomic sequence can lead to the production of multiple, related mRNAs and proteins. Both resulting mRNAs and proteins are referred to herein as “splice variants”.

The term “polypeptide” is used interchangeably herein with the terms “polypeptides” and “protein(s)”.

The term “variant” of a polypeptide refers to an amino acid sequence that is altered by one or more amino acids. The variant can have “conservative” changes, wherein a substituted amino acid has similar structural or chemical properties, e.g., replacement of leucine with isoleucine. Alternatively, a variant can have “non-conservative” changes, e.g., replacement of a glycine with a tryptophan. Analogous minor variation can also include amino acid deletion or insertion, or both. A particular form of a “variant” polypeptide is a “functionally equivalent” polypeptide, i.e., a polypeptide which exhibits substantially similar in vivo or in vitro activity as the naturally occurring endogenous polypeptide as described in more detail below. Guidance in determining which amino acid residues can be substituted, inserted, or deleted without eliminating biological or immunological activity can be found using computer programs well-known in the art, for example, DNASTAR software.

As used herein, a “chemical derivative” of a polypeptide of the invention is a polypeptide of the invention that contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16^{th} edition, Mack Publishing Co., Easton, Pa. (1980). The moieties may also be utilized to facilitate purification and identification of the claimed polypeptide.

The term “soluble TRP-1 protein or construct” refers to polypeptides, or substantially equivalent analogs, comprising an amino acid sequence corresponding to all or part of the extracellular region of a native TRP-1, or amino acid sequences substantially similar to the sequence of amino acids 1-136 of SEQ ID NO: 1, and which are biologically active in that they bind to molecules which bind to complete TRP-1 protein. The term “pegylated protein” refers to a protein, fragment thereof, or splice variant of a protein thereof having biological activity, which is covalently linked to a polyethylene glycol (PEG) group.

The term “polyethylene glycol” or “PEG” refers to a polyalkylene glycol compound or a derivative thereof, with or without coupling agents or derivatization with coupling or activating moieties, e.g., with thiol, triflate, tresylate, azirdine, oxirane, or preferably with a maleimide moiety. Other polyalkylene compounds include, but are not limited to, polypropylene glycol, charged or neutral polymers of dextran, colominic acids, or other carbohydrate based polymers, polymers of amino acids and biotin derivatives.

As used herein, the term “molecule” that binds to TRP-1 polypeptide or TRP-1 nucleic acid refers to any naturally occurring or chemically synthesized molecule that binds TRP-1 protein, variants or fragments thereof, splice variants of TRP-1 protein, and TRP-1 nucleic acid including, but not limited to, biological or synthetic organic compounds, inorganic compounds, proteins or fragments thereof, e.g., native ligands of TRP-1, antibodies or fragments thereof, toxins, carbohydrates and the like. The molecule may or may not modulate the activity of the TRP-1 protein, fragments thereof, or splice variants of TRP-1 protein, or transcription or translation of TRP-1 nucleic acid.

The term “therapeutic molecule” refers to any molecule that binds to TRP-1 protein, fragments thereof, splice variants of TRP-1 protein, or TRP-1 nucleic acid which is utilized to treat disorders including, but not limited to, inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders. Such a molecule includes, but is not limited to, biological or synthetic organic compounds, inorganic compounds, proteins or fragments thereof, e.g., native ligands of TRP-1, antibodies or fragments thereof, toxins and carbohydrates.

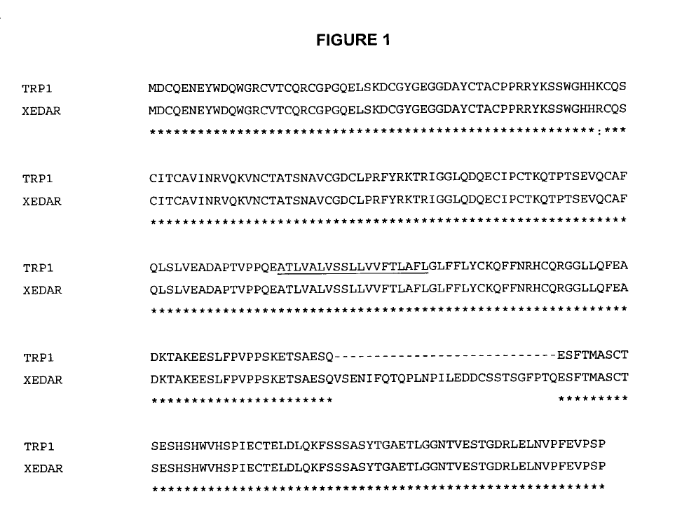

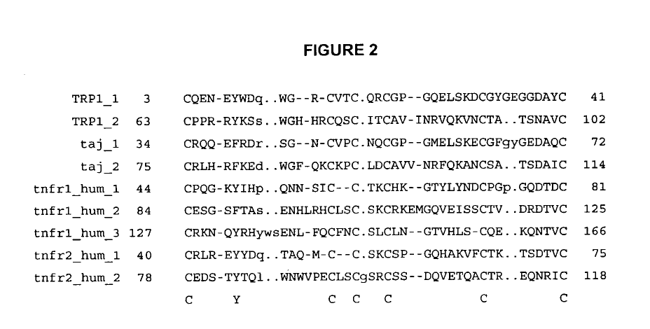

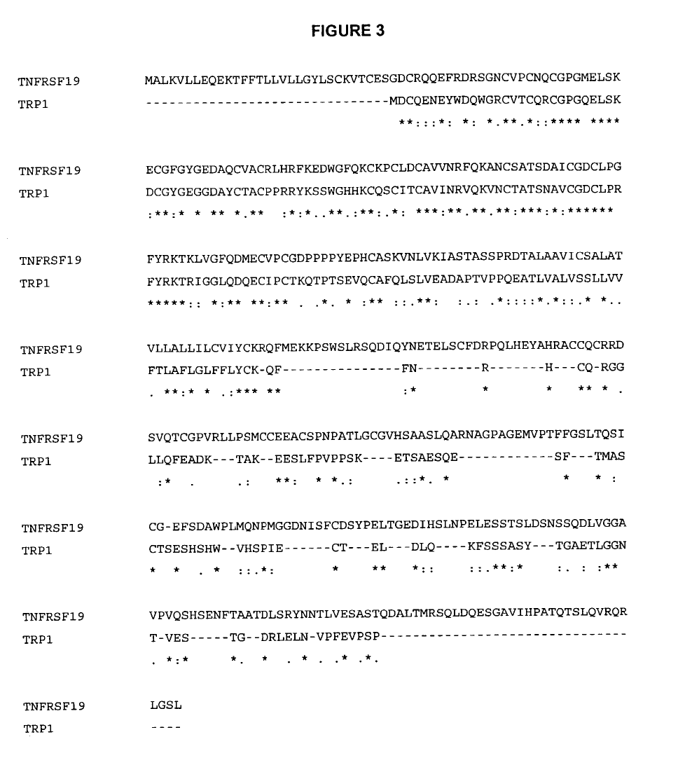

The invention generally relates to a novel nucleotide sequence which uniquely encodes a novel tumor necrosis factor receptor. The new gene encodes a polypeptide designated herein as tumor necrosis factor receptor related protein-1 or TRP-1, belonging to the TNFRSF as will be described in detail herein. TRP-1 is a splice variant of another TNFRSF member, XEDAR (Accession No. NM-021783, identified by Yan et al., Science, Vol. 290, pp. 523-527 (2000)), since it has a deletion of 28 amino acids in the intracellular domain and the bounds of this putative exon conform to the consensus splice site pattern GT/AG. FIG. 1 depicts the alignment of the peptide sequences of TRP-1 and XEDAR. The full-length cDNA for TRP-1 is set forth in SEQ ID NO: 2 (the open reading frame (ORF) is bp 113-922 including the stop codon). The deduced amino acid sequence (269 amino acids) for TRP-1 is set forth in SEQ ID NO: 1. The genomic DNA sequence for TRP-1 is set forth in SEQ ID NO: 3. Evidence support the assignment of TRP-1 as belonging to the TNFRSF. Sequence analyses using various search algorithms indicate that the predicted amino acid sequence of TRP-1 shows homology to the TNFRSF. Hidden Markov model analysis identified two cysteine-rich domains similar to the so called cysteine-rich pseudorepeats present in the extracellular domain of several TNFRSF members (see FIG. 2). Similar to other members of the TNFRSF, TRP-1 possesses a cysteine-rich motif in the extracellular domain, a highly hydrophobic transmembrane domain and a highly divergent putative cytoplasmic domain. The protein sequence of TRP-1 is most similar to that of TNFRSF19 (TROY, TAJ, see FIG. 3). Accordingly, TRP-1 likely transduces extracellular signals into a cell utilizing similar cellular signalling mechanisms as other TNFRSF members. As thus being qualified as a member of the TNFR family, the novel polypeptide TRP-1 will have similar physiological functions. The TRP-1 protein is likely to be a potent regulator of inflammation, immunity, cell proliferation, cell differentiation, viral replication, neurodegeneration, bone growth, hematopoiesis and apoptosis, due to its similarity to the TNFRSF receptors of known function, all of which have major impact in various human disorders including, but not limited to, inflammatory disorders, such as rheumatoid arthritis, osteoarthritis, Crohn's disease, acquired immunodeficiency syndrome (AIDS), Addison's disease, allergies, asthma, emphysema, bronchitis, irritable bowel syndrome, ulterative colitis, urethritis, multiple sclerosis, diabetes mellitus, thyroiditis, septic shock, septicemia; immune and autoimmune disorders, such as systemic lupus erythematosus and rheumatoid arthritis; neurodegenerative disorders, such as Alzheimer's disease, HIV-dementia and Parkinson's disease; cell proliferative disorders, such as polyps and various types of cancer, e.g., leukemia, lymphoma, sarcoma, adenocarcinoma and cancers of the breast, bone, brain, colon, heart, kidney, stomach, intestine, prostate, lung, ovary, skin, testis, thyroid, tongue, parathyroid and uterus; cell differentiation disorders, e.g., cancer; apoptotic disorders, such as accelerated aging, AIDS, neurodegenerative disease, ischemic injury, aplastic anemia, stroke and myocardial infarction; gastrointestinal and reproductive tract disorders, such as ulcers and prostatitis; bone disorders, such as osteoporosis; blood disorders, such as hematopoietic disorders, e.g., anemia including macrocytic and aplastic anemia and thrombocytopenia, viral disorders, such as viral infections, e.g., hepatitis, retroviral infections and viral encephalitis. The sequence and predicted structure of TRP-1 is useful for designing various-agents, e.g., anti-inflammatory agents either through the use of small molecules or proteins (e.g., antibodies) directed against it. In addition, protein derived from the TRP-1 sequence, e.g., soluble TRP-1 constructs and pegylated versions of TRP-1 as described below, may also be used as a therapeutic to treat such disorders, particularly inflammatory disorders.

The coding region of TRP-1 is distributed over 6 exons in a 16.5 kb region of chromosome X. This is the first TNFRSF member identified on this chromosome. Accordingly, the TRP-1 nucleic acid can be used as a marker for the X chromosome. Based on annotation of a public database of the human genome sequence the TRP-1 gene maps to band Xq12. To find possible diseases linked to the TRP-1 gene, a search of the OMIM™ Database (OMIM (2000)) for diseases mapped within Xq12-13 identified two diseases, Menkes disease and anhidrotic ectodermal dysplasia-1. Anhidrotic ectodermal dysplasia is a hereditary condition characterized by the abnormal development of skin, absence of sweat glands, dry eyes and abnormal development of teeth. Menkes disease is usually characterized by defective hair, neurological impairment and abnormal copper metabolism. Accordingly, the TRP-1 protein may also be involved in contributing to symptoms of these diseases.

To determine the expression pattern of the novel polypeptide, mRNA and cDNAs from a variety of human tissues are subjected to northern blot and RT-PCR analysis, respectively, using PCR primers that could identify TRP-1 transcript. PCR primers are chosen that are present on separate exons of the predicted TRP-1 gene so that PCR products from a transcribed, processed mRNA can readily be distinguished from any originating genomic DNA. As a result, RT-PCR shows that TRP-1 is differentially expressed in human tissues, being detectable in uterus, prostate, testis, and mammary gland, while Northern analysis shows that TRP-1 is highly expressed in the stomach. Thus, TRP-1 represents a transcribed gene.

Accordingly, in one aspect, the present invention relates to an isolated polypeptide comprising the amino acid sequence as set forth in SEQ ID NO: 1. Such a polypeptide may for example be a fusion protein including the amino acid sequence of the novel TRP-1 polypeptide. In another aspect the present invention relates to an isolated polypeptide consisting of the amino acid sequence set forth in SEQ ID NO: 1, which is, in particular, the novel TRP-1 polypeptide. In yet another aspect, the present invention relates to fragments of TRP-1 protein which may be constructed by deleting terminal or internal residues or sequences. Preferred amino acid sequences of TRP-1 include those in which the transmembrane and intracellular domains of the TRP-1 are deleted or substituted with hydrophilic residues to facilitate secretion of TRP-1 into cell culture medium. Such a TRP-1 protein is referred to as a soluble TRP-1 molecule or construct which retains its ability to bind molecules that bind full-length TRP-1 protein. A particularly preferred soluble TRP-1 construct is an isolated polypeptide which comprises the extracellular domain of TRP-1 as set forth in amino acids 1-136 of SEQ ID NO: 1. Additional amino acids may be deleted from the extracellular region while still retaining ligand binding activity.

In addition to TRP-1 soluble constructs, other fragments of TRP-1 can be generated from TRP-1 protein, such as fragments having a binding site, fragments having an epitope for antibody recognition (typically at least six amino acids) and fragments having only a transmembrane portion or a cytoplasmic portion. Such TRP-1 fragments can be used, e.g., to obtain antibodies to a particular region, select other molecules able to bind to full-length TRP-1 protein, and to form chimeric receptors with fragments of other receptors to create a new receptor having unique properties. Fragments of TRP-1 can be generated by standard techniques, such as expression of cloned partial sequences of TRP-1 DNA and proteolytic cleavage of TRP-1 proteins utilizing proteolytic enzymes, e.g., trypsin, chymotrypsin or pepsin.

The invention also includes various structural forms, i.e., chemical derivatives of TRP-1 polypeptide, variants, fragments or derivatives thereof, which retain biological activity. Such derivatives of TRP-1 can be generated, e.g., by forming covalent conjugates with other chemical groups including, but not limited to, phosphate, acetyl groups, glycosyl groups, etc. Covalent derivatives can be prepared by attaching particular functional groups to amino acid side chains of TRP-1 or to the N- or C-termini. Other derivatives of TRP-1 contemplated by the present invention include covalent conjugates of TRP-1 or fragments thereof with other proteins or peptides to form fusion proteins. For example, the conjugated peptide can be a signal peptide sequence at the N-terminus of the TRP-1 protein which when translated directs transfer of the protein to the outside of the cell membrane or to an intracellular organelle, e.g., the endoplasmic reticulum. TRP-1 fusion proteins can comprise peptides, e.g., poly-histidine, which can aid in purification or identification of TRP-1. Such TRP-1 fusion proteins are described in more detail below.

TRP-1 derivatives can also be utilized as immunogens, binding agents for affinity purification methods of molecules that bind TRP-1 proteins, fragments or splice variants thereof, and as reagents in receptor-based immunoassays. TRP-1 derivatives can also be obtained by reaction with cross-linking agents, e.g., N-maleimidobenzoyl succinimide ester and N-hydroxysuccinimide, which react with cysteine and lysine residues on TRP-1 polypeptide. TRP-1 proteins can also be covalently linked to various insoluble substrates, such as cyanogen bromide-activated, bisoxirane-activated or tosyl-activated agarose structures. When linked to such substrates, TRP-1 may be utilized to specifically bind antibodies or molecules that bind TRP-1 proteins.

TRP-1 derivatives also include TRP-1 polypeptides, fragments thereof, such as soluble TRP-1 constructs and splice variants of TRP-1 protein, covalently linked to polyalkylene oxide compounds, e.g., PEG to form pegylated versions of TRP-1. Modification of proteins by covalent attachment of polyalkylene oxide compounds increases the half-life of proteins after administration. Accordingly, such pegylated TRP-1 constructs, particularly soluble TRP-1 constructs, can be utilized to prevent or reduce TRP-1 dependent responses, e.g., inflammatory responses in a subject, by inactivating or regulating the activity of its native ligands. For example, U.S. Pat. No. 4,179,337, describes the use of PEG linked to proteins to provide a physiologically active non-immunogenic water soluble polypeptide composition. Nucci et al., Adv. Drug Delivery Rev., Vol. 4, pp. 133-151 (1991) describes the modification of several proteins including adenosine deamidase, L-asparaginase, interferon α2b, interleukins, epidermal growth factor and other growth factors by adding PEG. Typically, pegylation of proteins involves activating PEG with a functional group which will react with lysine residues on the surface of the protein. Recent developments in protein pegylation methods involve activating PEG with reagents which react with thiol groups of a protein, resulting in covalent linkage of PEG to a thiol group of a cysteine residue. The cysteine residue to which the activated PEG is attached is either present in the native protein or is introduced by site-specific mutation as described, e.g., in U.S. Pat. Nos. 5,766,897 and 5,166,322.

The invention includes isolated nucleic acid or nucleotide molecules, preferably DNA molecules, in particular encoding the novel TRP-1. Preferably, an isolated nucleic acid molecule, preferably a DNA molecule, of the present invention encodes a polypeptide comprising the amino acid sequence as set forth in SEQ ID NO: 1. Likewise preferred is an isolated nucleic acid molecule, preferably a DNA molecule, encoding a polypeptide consisting of the amino acid sequence set forth in SEQ ID NO: 1. Such a nucleic acid or nucleotide, in particular such a DNA molecule, preferably comprises a nucleotide sequence selected from the group consisting of:

(a) a nucleotide sequence that encodes a protein comprising the amino acid sequence set forth in SEQ ID NO: 1;

(b) a nucleotide sequence encoding amino acid residues 1-136 of SEQ ID NO: 1;

(c) a nucleotide sequence as set forth in nucleotides 113-922 of SEQ ID NO: 2;

(d) a nucleotide sequence as set forth in SEQ ID NO: 3;

(e) a nucleotide sequence capable of hybridizing under high stringency conditions to the nucleotide sequence set forth in nucleotides 113-922 SEQ ID NO: 2; and

(f) a nucleotide sequence which is complementary to the nucleotide sequence of (a), (b), (c), (d) or (e).

Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos) and 60° C. (for 23-base oligos). Suitable ranges of such stringency conditions for nucleic acids of varying compositions are described in Krause and Aaronson, Methods in Enzymology, Vol. 200, pp. 546-556 (1991), in addition to Maniatis et al., cited above.

These nucleic acid molecules may act as target gene anti-sense molecules, useful, for example, in target gene regulation and/or as anti-sense primers in amplification reactions of target gene nucleic acid sequences. Further, such sequences may be used as part of ribozyme and/or triple helix sequences, also useful for target gene regulation. Still further, such molecules may be used as components of diagnostic methods whereby the presence of an allele causing a disease, for example, one of the aforementioned disorders, may be detected.

The isolated nucleic acids can also comprise a coding region that encodes a splice variant of TRP-1 protein wherein the TRP-1 protein is encoded by a nucleotide sequence as set forth in SEQ ID NO: 3.

The invention also encompasses:

(a) vectors that contain at least a fragment of any of the foregoing nucleotide sequences and/or their complements (i.e., anti-sense);

(b) vector molecules, preferably vector molecules comprising transcriptional control sequences, in particular expression vectors, which contain any of the foregoing coding sequences operatively associated with a regulatory element that directs the expression of the coding sequences; and

(c) genetically engineered host cells that contain a vector molecule as mentioned herein or at least a fragment of any of the foregoing nucleotide sequences operatively associated with a regulatory element that directs the expression of the coding sequences in the host cell.

As used herein, “regulatory elements” include, but are not limited to, inducible and non-inducible promoters, enhancers, operators and other elements known to those skilled in the art that drive and regulate expression. Preferably, host cells can be vertebrate host cells, preferably mammalian host cells, like human cells, such as BHK cells; monkey cells, such as COS 1 or COS 7 cells; and rodent cells, such as CHO cells. Likewise preferred, host cells can be bacterial host cells, in particular E. coli cells.

Particularly preferred is a host cell, in particular of the above described type, which can be propagated in vitro and which is capable upon growth in culture of producing a TRP-1 polypeptide, in particular a polypeptide comprising or consisting of an amino acid sequence set forth in SEQ ID NO: 1, wherein the cell comprises at least one transcriptional control sequence that is not a transcriptional control sequence of the natural endogeneous human gene encoding the polypeptide, wherein one or more transcriptional control sequences control transcription of a DNA encoding the polypeptide.

The invention also includes fragments of any of the nucleic acid sequences disclosed herein. Fragments of the nucleic acid sequences encoding the novel TRP-1 polypeptide may be used as a hybridization probe for a cDNA library to isolate the full length gene and to isolate other genes which have a high sequence similarity to the TRP-1 gene or similar biological activity. Probes of this type preferably have at least about 30 bases and may contain, for example, from about 30 to about 50 bases, about 50 to about 100 bases, about 100 to about 200 bases or more than 200 bases. The probe may also be used to identify a cDNA clone corresponding to a full length transcript and a genomic clone or clones that contain the complete TRP-1 gene including regulatory and promoter regions, exons and introns. An example of a screen comprises isolating the coding region of the TRP-1 gene by using the known DNA sequence to synthesize an oligonucleotide probe. Labelled oligonucleotides having a sequence complementary to that of the gene of the present invention are used to screen a library of human cDNA, genomic DNA or mRNA to determine which members of the library the probe hybridizes to. Fragments of the nucleic acid sequence encoding TRP-1 protein can also be utilized to generate TRP-1 protein fragments, such as soluble TRP-1 protein constructs, as described above.

In addition to the gene sequences described above, homologs of such sequences, as may, for example, be present in other species, may be identified and may be readily isolated, without undue experimentation, by molecular biological techniques well-known in the art. Further, there may exist genes at other genetic loci within the genome that encode proteins which have extensive homology to one or more domains of such gene products. These genes may also be identified via similar techniques.

For example, the isolated nucleotide sequence of the present invention encoding the novel TRP-1 polypeptide may be labelled and used to screen a cDNA library constructed from mRNA obtained from the organism of interest. Hybridization conditions will be of a lower stringency when the cDNA library was derived from an organism different from the type of organism from which the labelled sequence was derived. Alternatively, the labelled fragment may be used to screen a genomic library derived from the organism of interest, again, using appropriately stringent conditions. Such low stringency conditions will be well known to those of skill in the art, and will vary predictably depending on the specific organisms from which the library and the labelled sequences are derived. For guidance regarding such conditions see, for example, Sambrook et al., supra.

Further, a previously unknown differentially expressed gene-type sequence may be isolated by performing PCR using two degenerate oligonucleotide primer pools designed on the basis of amino acid sequences within the gene of interest. The template for the reaction may be cDNA obtained by reverse transcription of mRNA prepared from human or non-human cell lines or tissue known or suspected to express a differentially expressed gene allele.

The PCR product may be sub-cloned and sequenced to ensure that the amplified sequences represent the sequences of a differentially expressed gene-like nucleic acid sequence. The PCR fragment may then be used to isolate a full-length cDNA clone by a variety of methods. For example, the amplified fragment may be labelled and used to screen a bacteriophage cDNA library. Alternatively, the labelled fragment may be used to screen a genomic library.

PCR technology may also be utilized to isolate full-length cDNA sequences. For example, RNA may be isolated, following standard procedures, from an appropriate cellular or tissue source. A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see, e.g., Sambrook et al., 1989, supra.

In cases where the gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to symptoms related to the aforementioned disorders. Mutant alleles and mutant allele products may then be utilized in the diagnostic assay systems described below.

A cDNA of the mutant gene may be isolated, for example, by using PCR, a technique which is well-known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well-known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labelled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. For screening techniques, see, for example, Harlow and Lane, eds., Antibodies: A Laboratory Manual, Cold Spring Harbor Press, Cold Spring Harbor (1988). In cases where the mutation results in an expressed gene product with altered function (e.g., as a result of a missense mutation), a polyclonal set of antibodies are likely to cross-react with the mutant gene product. Library clones detected via their reaction with such labelled antibodies can be purified and subjected to sequence analysis as described above.

The present invention also includes those proteins encoded by the nucleotide sequence set forth in nucleotides 113-922 of SEQ ID NO:2 and nucleotide sequence of SEQ ID NO: 3, in particular as stated above, a polypeptide that is or includes the amino acid sequence set out in SEQ ID NO: 1 or fragments thereof.

Furthermore, the present invention includes proteins that represent functionally equivalent gene products. Such an equivalent differentially expressed gene product may contain deletions, additions or substitutions of amino acid residues within the amino acid sequence encoded by the differentially expressed gene sequences described above, but which result in a silent change, thus producing a functionally equivalent differentially expressed gene product. Amino acid substitutions may be made on the basis of similarity in polarity, charge, solubility, hydrophobicity, hydrophilicity and/or the amphipathic nature of the residues involved.

For example, non-polar (hydrophobic) amino acids include alanine, leucine, isoleucine, valine, proline, phenylalanine, tryptophan and methionine; polar neutral amino acids include glycine, serine, threonine, cysteine, tyrosine, asparagine and glutamine; positively charged (basic) amino acids include arginine, lysine and histidine; and negatively charged (acidic) amino acids include aspartic acid and glutamic acid. “Functionally equivalent” as utilized herein, may refer to a protein or polypeptide capable of exhibiting a substantially similar in vivo or in vitro activity as the endogenous differentially expressed gene products encoded by the differentially expressed gene sequences described above. “Functionally equivalent” may also refer to proteins or polypeptides capable of interacting with other cellular or extracellular molecules in a manner substantially similar to the way in which the corresponding portion of the endogenous differentially expressed gene product would. For example, a “functionally equivalent” peptide would be able, in an immunoassay, to diminish the binding of an antibody to the corresponding peptide (i.e., the peptide the amino acid sequence of which was modified to achieve the “functionally equivalent” peptide) of the endogenous protein, or to the endogenous protein itself, where the antibody was raised against the corresponding peptide of the endogenous protein. An equimolar concentration of the functionally equivalent peptide will diminish the aforesaid binding of the corresponding peptide by at least about 5%, preferably between about 5% and 10%, more preferably between about 10% and 25%, even more preferably between about 25% and 50%, and most preferably between about 40% and 50%.

The polypeptides of the present invention may be produced by recombinant DNA technology using techniques well-known in the art. Therefore, there is provided a method of producing a polypeptide of the present invention, which method comprises culturing a host cell having incorporated therein an expression vector containing an exogenously-derived polynucleotide encoding a polypeptide comprising an amino acid sequence as set forth in SEQ ID NO: 1 under conditions sufficient for expression of the polypeptide in the host cell, thereby causing the production of the expressed polypeptide. Optionally, the method further comprises recovering the polypeptide produced by the cell. In a preferred embodiment of such a method, the exogenously-derived polynucleotide encodes a polypeptide consisting of an amino acid sequence set forth in SEQ ID NO: 1. Preferably, the exogenously-derived polynucleotide comprises the nucleotide sequence as set forth in nucleotides 113-922 of SEQ ID NO: 2 and the nucleotide sequence of SEQ ID NO: 3. In case of using the nucleotide sequence set forth in nucleotides 113-922 of SEQ ID NO: 2, i.e., the ORF, the sequence, when inserted into a vector, may be followed by one or more appropriate translation stop codons.

Thus, methods for preparing the polypeptides and peptides of the invention by expressing nucleic acid encoding respective nucleotide sequences are described herein. Methods which are well-known to those skilled in the art can be used to construct expression vectors containing protein coding sequences and appropriate transcriptional/translational control signals. These methods include, for example, in vitro recombinant DNA techniques, synthetic techniques and in vivo recombination/genetic recombination (see, e.g., the techniques described in Sambrook et al., 1989, supra; and Ausubel et al., 1989, supra.). Alternatively, RNA capable of encoding differentially expressed gene protein sequences may be chemically synthesized using, for example, synthesizers (see, e.g., the techniques described in Oligonucleotide Synthesis, Gait, ed., IRL Press, Oxford (1984)).

A variety of host-expression vector systems may be utilized to express the differentially expressed gene coding sequences of the invention. Such host-expression systems represent vehicles by which the coding sequences of interest may be produced and subsequently purified, but also represent cells which may, when transformed or transfected with the appropriate nucleotide coding sequences, exhibit the differentially expressed gene protein of the invention in situ. These include but are not limited to microorganisms, such as bacteria, e.g., E. coli, B. subtilis, transformed with recombinant bacteriophage DNA, plasmid DNA or bacterial artificial chromosome (BAC) DNA expression vectors containing differentially expressed gene protein coding sequences:, yeast, e.g., Saccharomyces, Pichia, transformed with recombinant yeast expression vectors containing the differentially expressed gene protein coding sequences; insect cell systems infected or transfected with recombinant virus expression vectors, e.g., baculovirus, containing the differentially expressed gene protein coding sequences; plant cell systems infected with recombinant virus expression vectors, e.g., cauliflower mosaic virus (CaMV) and tobacco mosaic virus (TMV); or transformed with recombinant vectors, including plasmids, e.g., Ti plasmid, containing protein coding sequences; or mammalian cell systems, e.g., COS, CHO, BHK, 293, 3T3, harboring recombinant expression constructs containing promoters derived from the genome of mammalian cells, e.g., metallothionein promoter; or from mammalian viruses, e.g., the adenovirus late promoter; the vaccinia virus 7.5K promoter or the CMV promoter.

Expression of the TRP-1 polypeptide of the present invention by a cell from a TRP-1 encoding gene that is native to the cell can also be performed. Methods for such expression are detailed in, e.g., U.S. Pat. Nos. 5,641,670; 5,733,761; and 5,968,502. Cells that have been induced to express TRP-1 by the methods of any of U.S. Pat. Nos. 5,641,670; 5,733,761; and 5,968,502; can be implanted into a desired tissue in a living animal in order to increase the local concentration of TRP-1 in the tissue.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable. In this respect, fusion proteins comprising hexahistidine tags may be used as described, e.g., Porath, Protein Express. Purif., Vol. 3, pp. 263-281 (1992); Yang et al., Amer. Biotechnol Lab., pp. 12-14 (1997); and Kasher et al., BioTechniques, Vol. 14, pp. 630-641 (1993). Such vectors include, but are not limited, to the E. coli expression vector pUR278 (see Ruther et al., EMBO, J. 2, p. 1791 (1983)), in which the protein-encoding sequence may be ligated individually into the vector in frame with the lac Z coding region so that a fusion protein is produced; pIN vectors (see Inouye & Inouye, Nucl. Acids Res., Vol. 13, pp. 3101-3109 (1985); Van Heeke & Schuster, J. Biol. Chem., Vol. 264, pp. 5503-5509 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

Promoter regions can be selected from any desired gene using vectors that contain a reporter transcription unit lacking a promoter region, such as a chloramphenicol acetyl transferase (“CAT”), or the luciferase transcription unit, downstream of restriction site or sites for introducing a candidate promoter fragment; i.e., a fragment that may contain a promoter. For example, introduction into the vector of a promoter-containing fragment at the restriction site upstream of the cat gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well-known and readily-available promoters, but also promoters that readily may be obtained by the foregoing technique, using a reporter gene.

Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”); and metallothionein promoters, such as the mouse metallothionein-I promoter.

In an insect system, Autographa californica nuclear polyhedrosis virus (AcNPV) is one of several insect systems that can be used as a vector to express foreign genes. The virus grows in Spodoptera frugiperda cells. The coding sequence may be cloned individually into non-essential regions, for example, the polyhedrin gene, of the virus and placed under control of an AcNPV promoter, for example, the polyhedrin promoter. Successful insertion of the coding sequence will result in inactivation of the polyhedrin gene and production of non-occluded recombinant virus, i.e., virus lacking the proteinaceous coat coded for by the polyhedrin gene. These recombinant viruses are then used to infect Spodoptera frugiperda cells in which the inserted gene is expressed (see, e.g., Smith et al., J. Virol., Vol. 46, p. 584 (1983); and Smith, U.S. Pat. No. 4,215,051).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome, e.g., region E1 or E3, will result in a recombinant virus that is viable and capable of expressing the desired protein in infected hosts (see, e.g., Logan & Shenk, Proc. Natl. Acad. Sci. USA, Vol. 81, pp. 3655-3659 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation, codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol., Vol. 153, pp. 516-544 (1987)). Other common systems are based on SV40, retrovirus or adeno-associated virus. Selection of appropriate vectors and promoters for expression in a host cell is a well-known procedure and the requisite techniques for expression vector construction, introduction of the vector into the host and expression in the host per se are routine skills in the art. Generally, recombinant expression vectors will include origins of replication, a promoter derived from a highly-expressed gene to direct transcription of a downstream structural sequence, and a selectable marker to permit isolation of vector containing cells after exposure to the vector.

In addition, a host cell strain may be chosen which modulates the expression of the inserted sequences, or modifies and processes the gene product in the specific fashion desired. Such modifications, e.g., glycosylation, and processing, e.g., cleavage, of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation and phosphorylation of the gene product may be used. Such mammalian host cells include, but are not limited to, CHO, VERO, BHK, HeLa, COS, MDCK, 293, 3T3, W138, etc.

For long-term, high-yield production of recombinant proteins, stable expression is preferred. For example, cell lines which stably express the differentially expressed gene protein may be engineered. Rather than using expression vectors which contain viral origins of replication, host cells can be transformed with DNA controlled by appropriate expression control elements, e.g., promoter, enhancer, sequences, transcription terminators, polyadenylation sites, etc., and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed gene protein. Such engineered cell lines may be particularly useful in screening and evaluation of compounds that affect the endogenous activity of the expressed protein.

A number of selection systems may be used including, but not limited to, the herpes simplex virus thymidine kinase (see Wigler et al., Cell, Vol. 11, p. 223 (1977)); hypoxanthine-guanine phosphoribosyltransferase (see Szybalska & Szybalski, Proc. Natl. Acad. Sci. USA, Vol. 48, p. 2026 (1962)); and adenine phosphoribosyltransferase (see Lowy et al., Cell, Vol. 22, p. 817 (1980)) genes can be employed in tk⁻, hgprt⁻ or aprt⁻ cells, respectively. Also, anti-metabolite resistance can be used as the basis of selection for dhfr, which confers resistance to methotrexate (see Wigler et al., Proc. Natl. Acad. Sci. USA, Vol. 77, p. 3567 (1980); O'Hare et al., Proc. Natl. Acad. Sci. USA, Vol. 78, p. 1527 (1981,)); gpt, which confers resistance to mycophenolic acid (see Mulligan & Berg, Proc. Natl. Acad. Sci. USA, Vol. 78, p. 2072 (1981)); neo, which confers resistance to the aminoglycoside G-418 (see Colberre-Garapin et al., J. Mol. Biol., Vol. 150, p. 1 (1981)); and hygro, which confers resistance to hygromycin genes (see Santerre et al., Gene, Vol. 30, p. 147 (1984)).

An alternative fusion protein system allows for the ready purification of non-denatured fusion proteins expressed in human cell lines (see Janknecht et al., Proc. Natl. Acad. Sci. USA, Vol. 88, pp. 8972-8976 (1991)). In this system, the gene of interest is subcloned into a vaccinia recombination plasmid such that the gene's ORF is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, a protein of the present invention may be labelled, either directly or indirectly, to facilitate detection of a complex formed between the protein and a test substance. Any of a variety of suitable labelling systems may be used including, but not limited to, radioisotopes, such as ¹²⁵I; enzyme labelling systems that generate a detectable calorimetric signal or light when exposed to substrate and fluorescent labels.

Where recombinant DNA technology is used to produce a protein of the present invention for such assay systems, it may be advantageous to engineer fusion proteins that can facilitate labelling, immobilization, detection and/or isolation.

Indirect labelling involves the use of a protein, such as a labelled antibody, which specifically binds to a polypeptide of the present invention. Such antibodies include, but are not limited to, polyclonal, monoclonal, chimeric, single-chain, Fab fragments and fragments produced by an Fab expression library.

In another embodiment, nucleic acids comprising a sequence encoding a TRP-1 protein or functional derivative thereof, are administered to reduce TRP-1 functions, e.g., host inflammatory response, immune response, apoptosis, etc., by way of gene therapy. Gene therapy refers to therapy performed by the administration of a nucleic acid to a subject. In this embodiment of the invention, the nucleic acid produces its encoded protein that mediates a therapeutic effect by reducing TRP-1 function.

Any of the methods for gene therapy available in the art can be used according to the present invention. Exemplary methods are described below.

In a preferred aspect, the therapeutic comprises a TRP-1 nucleic acid that is part of an expression vector that expresses a TRP-1 protein or fragment or chimeric protein thereof in a suitable host. In particular, such a nucleic acid has a promoter operably-linked to the TRP-1 coding region, the promoter being inducible or constitutive, and, optionally, tissue-specific. In another particular embodiment, a nucleic acid molecule is used in which the TRP-1 coding sequences and any other desired sequences are flanked by regions that promote homologous recombination at a desired site in the genome, thus providing for intrachromosomal expression of the TRP-1 nucleic acid (see Koller and Smithies, Proc. Natl. Acad. Sci. USA, Vol. 86, pp. 8932-8935 (1989); Zijlstra et al., Nature, Vol. 342, pp. 435-438 (1989)).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment, e.g., a gene gun; Biolistic, Dupont), or coating with lipids or cell-surface receptors or transfecting agents, encapsulation in liposomes, microparticles or microcapsules, or by administering it in linkage to a peptide which is known to enter the nucleus, by administering it in linkage to a ligand subject to receptor-mediated endocytosis (see, e.g., U.S. Pat. Nos. 5,166,320; 5,728,399; 5,874,297; and 6,030,954) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., Nature, Vol. 342, pp. 435-438 (1989)).

In a specific embodiment, a viral vector that contains the TRP-1 nucleic acid is used. For example, a retroviral vector can be used (see, e.g., U.S. Pat. No. 5,219,740). These retroviral vectors have been modified to delete retroviral sequences that are not necessary for packaging of the viral genome and integration into host cell DNA. The TRP-1 nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,880,102; 5,882,877; 5,885,808; 5,932,210; 5,981,225; 5,994,106; 5,994,132; 5,994,134; and 6,001,557.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,773,289; 5,843,742; 5,869,040; 5,604,090; 5,834,182; and 5,948,675.

Another approach to gene therapy involves transferring a gene to cells in tissue culture by such methods as electroporation, lipofection, calcium phosphate mediated transfection or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells, e.g., hematopoietic stem or progenitor cells, are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type and include, but are not limited to, epithelial cells, endothelial cells, keratinocytes, fibroblasts, muscle cells and hepatocytes; blood cells, such as T lymphocytes, B lymphocytes, monocytes, macrophages, neutrophils, eosinophils, megakaryocytes and granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a TRP-1 nucleic acid is introduced into the cells such that it is expressible by the cells or their progeny, and the recombinant cells are then administered in vivo for therapeutic effect. In a specific embodiment, stem or progenitor cells are used. Any stem-and/or progenitor cells which can be isolated and maintained in vitro can potentially be used in accordance with this embodiment of the present invention. Such stem cells include, but are not limited to, hematopoietic stem cells (HSC), stem cells of epithelial tissues such as the skin and the lining of the gut, embryonic heart muscle cells, liver stem cells (see, e.g., WO 94/08598), and neural stem cells (see Stemple and Anderson, Cell, Vol. 71, pp. 973-985 (1992)).

Epithelial stem cells (ESCs) or keratinocytes can be obtained from tissues, such as the skin and the lining of the gut by known procedures (see Rheinwald, Meth. Cell Bio., Vol. 21A, p. 229 (1980)). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (see Pittelkow and Scoff, Mayo Clinic Proc., Vol. 61, p. 771 (1986)). If the ESCs are provided by a donor, a method for suppression of host versus graft reactivity, e.g., irradiation, drug or antibody administration to promote moderate immunosuppression, can also be used.

With respect to hematopoietic stem cells (HSC), any technique which provides for the isolation, propagation and maintenance in vitro of HSC can be used in this embodiment of the invention. Techniques by which this may be accomplished include:

(a) the isolation and establishment of HSC cultures from bone marrow cells isolated from the future host, or a donor, or

(b) the use of previously established long-term HSC cultures, which may be allogeneic or xenogeneic.

Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., J. Clin. Invest., Vol. 73, pp. 1377-1384 (1984)). In a preferred embodiment of the present invention, the HSCs can be made highly-enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (see Dexter et al., J. Cell Physiol., Vol. 91, p. 335 (1977)) or Witlock-Witte culture techniques (see Witlock and Witte, Proc. Natl. Acad. Sci. USA, Vol. 79, pp. 3608-3612 (1982)).

In a specific embodiment, the nucleic acid to be introduced for purposes of gene therapy comprises an inducible promoter operably-linked to the coding region, such that expression of the nucleic acid is controllable by controlling the presence or absence of the appropriate inducer of transcription.

A further embodiment of the present invention relates to a purified antibody or a fragment thereof which specifically binds to a polypeptide that comprises the amino acid sequence set forth in SEQ ID NO: 1 or to a fragment of the polypeptide. A preferred embodiment relates to a fragment of such an antibody, which fragment is an Fab or F(ab′)₂ fragment. In particular, the antibody can be a polyclonal antibody or a monoclonal antibody.

Described herein are methods for the production of antibodies capable of specifically recognizing one or more differentially expressed gene epitopes. Such antibodies may include, but are not limited to, polyclonal antibodies, monoclonal antibodies (mAbs), humanized or chimeric antibodies, single-chain antibodies, Fab fragments, F(ab′)₂ fragments, fragments produced by a Fab expression library, anti-idiotypic (anti-Id) antibodies and epitope-binding fragments of any of the above. Such antibodies may be used, for example, in the detection of a fingerprint, target, gene in a biological sample, or, alternatively, as a method for the inhibition of abnormal target gene activity. Thus, such antibodies may be utilized as part of a disease treatment method, and/or may be used as part of diagnostic techniques whereby patients may be tested for abnormal levels of the TRP-1 polypeptide, or for the presence of abnormal forms of the TRP-1 polypeptide.

For the production of antibodies to the TRP-1 polypeptide, various host animals may be immunized by injection with the TRP-1 polypeptide, or a portion thereof. Such host animals may include, but are not limited to, rabbits, mice and rats, to name but a few. Various adjuvants may be used to increase the immunological response, depending on the host species including, but not limited to, Freund's (complete and incomplete), mineral gels, such as aluminum hydroxide; surface active substances, such as lysolecithin, pluronic polyols, polyanions, peptides, oil emulsions, keyhole limpet hemocyanin, dinitrophenol; and potentially useful human adjuvants, such as bacille Calmette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the TRP-1 polypeptide, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, Nature, Vol. 256, pp. 495-497 (1975); and U.S. Pat. No. 4,376,110, the human B-cell hybridoma technique (see Kosbor et al., Immunology Today, Vol. 4, p. 72 (1983); Cole et al., Proc. Natl. Acad. Sci. USA, Vol. 80, pp. 2026-2030 (1983)) and the EBV-hybridoma technique (see Cole et al., Monoclonal Antibodies and Cancer Therapy, Alan R. Liss, Inc., pp. 77-96 (1985)). Such antibodies may be of any immunoglobulin class including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (see Morrison et al., Proc. Natl. Acad. Sci. USA, Vol. 81, pp. 6851-6855 (1984); Neuberger et al., Nature, Vol. 312, pp. 604-608 (1984); and Takeda et al., Nature, Vol. 314, pp. 452-454 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, Science, Vol. 242, pp. 423-426 (1988); Huston et al., Proc. Nat. Acad. Sci. USA, Vol. 85, pp. 5879-5883 (1988); and Ward et al., Nature, Vol. 334, pp. 544-546 (1989)) can be adapted to produce differentially expressed gene-single-chain antibodies. Single-chain antibodies are formed by linking the heavy and light chain fragments of the Fv region via an amino acid bridge, resulting in a single-chain polypeptide.

Most preferably, techniques useful for the production of “humanized antibodies” can be adapted to produce antibodies to the polypeptides, fragments, derivatives and functional equivalents disclosed herein. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (see Huse et al., Science, Vol. 246, pp. 1275-1281 (1989)) to allow rapid and easy identification of monoclonal Fab fragments with the desired specificity.

An antibody of the present invention can be preferably used in a method for the diagnosis of disorders selected from the group consisting of inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders in a human, the method comprising detecting the amount of a polypeptide comprising the amino acid sequence set forth in SEQ ID NO: 1, or fragments thereof, in an appropriate tissue or cell from a human suffering from a such a disorder, wherein the presence of an elevated or reduced amount of the polypeptide or fragments thereof, relative to the amount of the polypeptide or fragments thereof in the respective tissue from a human not suffering from such a disorder is diagnostic of the human's suffering from such a disorder. Such a method forms a further embodiment of the present invention. Preferably, the detecting step comprises contacting the appropriate tissue or cell with an antibody which specifically binds to a polypeptide that comprises the amino acid sequence set forth in SEQ ID NO: 1 or a fragment thereof and detecting specific binding of the antibody with a polypeptide in the appropriate tissue or cell, wherein detection of specific binding to a polypeptide indicates the presence of a polypeptide that comprises the amino acid sequence set forth in SEQ ID NO: 1 or a fragment thereof.

Particularly preferred, for ease of detection, is the sandwich assay, of which a number of variations exist, all of which are intended to be encompassed by the present invention.

For example, in a typical forward assay, unlabeled antibody is immobilized on a solid substrate. The sample to be tested is then brought into contact with the bound molecule and incubated for a period of time sufficient to allow formation of an antibody-antigen binary complex. At this point, a second antibody, labelled with a reporter molecule capable of inducing a detectable signal, is then added and incubated, allowing time sufficient for the formation of a ternary complex of antibody-antigen-labelled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labelled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labelled antibody be an antibody which is specific for the TRP-1 polypeptide or a fragment thereof.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of TRP-1 which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

This invention also relates to the use of polynucleotides of the present invention as diagnostic reagents. In particular, the invention relates to a method for the diagnosis of a disorder selected from the group consisting of inflammatory disorders, immune disorders, neurodegenerative disorders, cell proliferative disorders, cell differentiation disorders, apoptotic disorders, gastrointestinal and reproductive tract disorders, bone disorders, blood disorders and viral disorders in a human, the method comprising detecting elevated or reduced transcription of mRNA transcribed from the natural endogeneous human gene encoding the polypeptide consisting of an amino acid sequence set forth in SEQ ID NO: 1 in an appropriate tissue or cell from a human, wherein the elevated or reduced transcription is diagnostic of the human's suffering from the disorder. In particular, the natural endogeneous human gene comprises the nucleotide sequence set forth in SEQ ID NO: 3. In a preferred embodiment such a method comprises contacting a sample of the appropriate tissue or cell or contacting an isolated RNA or DNA molecule derived from that tissue or cell with an isolated nucleotide sequence of at least about 20 nucleotides in length that hybridizes under high stringency conditions with the isolated nucleotide sequence encoding a polypeptide consisting of an amino acid sequence set forth in SEQ ID NO: 1.

Detection of a mutated form of the gene characterized by the polynucleotide of SEQ ID NO: 3 which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labelled nucleotide sequences encoding the TRP-1 polypeptide of the present invention. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (see, e.g., Myers et al., Science, Vol. 230, p. 1242 (1985)). Sequence changes at specific locations may also be revealed by nuclease protection assays, such as RNase and S1 protection or the chemical cleavage method (see Cotton et al., Proc. Natl. Acad. Sci. USA, Vol. 85, pp. 4397-4401 (1985)). In another embodiment, an array of oligonucleotides probes comprising nucleotide sequence encoding the TRP-1 polypeptide of the present invention or fragments of such a nucleotide sequence can be constructed to conduct efficient screening of, e.g., genetic mutations. Array technology methods are well known and have general applicability and can be used to address a variety of questions in molecular genetics including gene expression, genetic linkage, and genetic variability (see, e.g., Chee et al., Science, Vol. 274, pp. 610-613 (1996)).

The diagnostic assays offer a process for diagnosing or determining a susceptibility to disease such as the aforementioned disorders, through detection of mutation in the TRP-1 gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit which comprises:

(a) a polynucleotide of the present invention, preferably the nucleotide sequence as set forth in nucleotides 113-922 of SEQ ID NO: 2 and the nucleotide sequence of SEQ ID NO: 3 or a fragment thereof;

(b) a nucleotide sequence complementary to that of (a);

(c) a polypeptide of the present invention, preferably the polypeptide of SEQ ID NO: 1 or a fragment thereof; or

(d) an antibody to a polypeptide of the present invention, preferably to the polypeptide of SEQ ID NO: 1.

It will be appreciated that in any such kit, (a), (b), (c) or (d) may comprise a substantial component. Such a kit will be of use in diagnosing a disease, e.g., one of the aforementioned disorders or the susceptibility to such a disorder.

The nucleotide sequences of the present invention are also valuable for chromosome localization. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, e.g., McKusick, “Mendelian Inheritance in Man”, available on-line through National Center for Biotechnology Information. The relationship between genes and diseases that have been mapped to the same chromosomal region are then identified through linkage analysis (co-inheritance of physically adjacent genes).

The differences in the cDNA or genomic sequence between affected and unaffected individuals can also be determined. If a mutation is observed in some or all of the affected individuals but not in any normal individuals, then the mutation is likely to be a causative agent of the disease.

An additional embodiment of the invention relates to the administration of a pharmaceutical composition, in conjunction with a pharmaceutically acceptable carrier, excipient or diluent, for any of the therapeutic effects discussed above. Such pharmaceutical compositions comprise an effective amount of the TRP-1 polypeptide, variants or analogs thereof, fragments of the TRP-1 polypeptide or variants, splice variants of the TRP-1 polypeptide, antibodies to that polypeptide, mimetics, agonists, antagonists or inhibitors of TRP-1 function. In a preferred embodiment, the pharmaceutical composition comprises an effective amount of a soluble TRP-1 protein construct, or a pegylated TRP-1 protein, particularly a pegylated soluble TRP-1 protein construct as described above, in combination with a pharmaceutically acceptable carrier. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone, or in combination with other agents, drugs or hormones.

The pharmaceutical compositions encompassed by the invention may be administered by any number of routes including, but not limited to, oral, intravenous, intramuscular, intraarticular, intraarterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences, Maack Publishing Co., Easton, Pa.

Pharmaceutical compositions for oral administration can be formulated using pharmaceutically-acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid or liquid polyethylene glycol with or without stabilizers.

Pharmaceutical formulations suitable for parenteral administration may be formulated in aqueous solutions, preferably in physiologically compatible buffers such as Hanks' solution, Ringer's solution or physiologically buffered saline. Aqueous injection suspensions may contain substances which increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1-2% sucrose, and 2-7% mannitol, at a pH range of 4.5-5.5, that is combined with buffer prior to use.

After pharmaceutical compositions have been prepared, they can be placed in an appropriate container and labelled for treatment of an indicated condition. For administration of the TRP-1 protein, such labelling would include amount, frequency and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

A therapeutically effective dose refers to that amount of active ingredient, for example, soluble TRP-1, antibodies or fragments thereof of TRP-1, agonists, antagonists or inhibitors of TRP-1, which ameliorates the symptoms or condition. Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight and gender of the subject, diet, time and frequency of administration, drug combination(s), reaction sensitivities and tolerance/response to therapy. Long-acting pharmaceutical compositions may be administered every 3-4 days, every week or once every two weeks depending on half-life and clearance rate of the particular formulation.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, the invention is directed to methods of identifying molecules that can bind to and/or modulate the activity of a TRP-1 polypeptide comprising or consisting of an amino acid sequence set forth in SEQ ID NO: 1. The method comprises:

a) contacting an isolated cell which expresses a heterologous nucleic acid encoding the TRP-1 polypeptide with the molecule of interest; and

b) determining binding and/or modulation of the TRP-1 polypeptide by the molecule, to identify a molecule which binds with or modulates the activity of the TRP-1 polypeptide.

Cell-free assays can also be used to identify molecules which are capable of binding with the TRP-1 protein. Such cell-free assays generally involve incubating the TRP-1 protein with the candidate molecule and determining the activity of the TRP-1 protein.

In yet another aspect, the invention is directed to methods of identifying molecules that can bind to and/or modulate the transcription or translation of a heterologous nucleic acid encoding a TRP-1 polypeptide comprising or consisting of an amino acid sequence as set forth in SEQ ID NO: 1. The method comprises:

a) contacting an isolated cell comprising the nucleic acid with the molecule; and

b) determining binding of the nucleic acid and/or modulation of the transcription or translation of the nucleic acid by the molecule, to identify molecules which bind to and/or modulate the transcription or translation of the nucleic acid.

The ability of a molecule to bind and/or modulate the activity of the expressed TRP-1 protein, or the ability of a molecule to bind to a nucleic acid encoding a TRP-1 protein and/or modulate the transcription or translation of a TRP-1 nucleic acid can be determined by methods well-known to those skilled in the art.

For example, once identified as a TNF receptor like molecule the expressed TRP-1 protein readily allows for identification of molecules that bind to the TRP-1 protein, e.g., proteins, such as counterreceptors and ligands, via expression cloning strategies. Two properties of TNF receptors and ligands make this possible. First, it is well-known that TNF receptors can be expressed as soluble secreted forms that retain their ability to bind counter receptors and ligands. Second, proteins that bind to TNF receptors are made as secreted and/or membrane bound forms. Membrane bound forms of molecules, e.g., proteins, that bind to TRP-1 protein thus can be identified by using a labelled secreted form of TRP-1 and screening either a bacterial expression library or mammalian cells transfected with a mammalian cDNA expression library. Cells expressing a molecule, e.g., a protein, that binds to TRP-1 protein can readily be detected when using a TRP-1 protein with either an epitope tag for which a detection agent is available or using radioactive or otherwise labelled TRP-1 protein. In addition, soluble TRP-1 protein can also be used to isolate and identify molecules that bind to TRP-1 protein by affinity chromotagraphy or other expression cloning techniques as described above or in other references (see, e.g., Davis et al., Cell, Vol. 87, No. 7, pp. 1161-1169 (1996)).

An example of a method to identify molecules that can lead to a decrease in the level of transcription of the TRP-1 mRNA involves identifying the promoter regions involved in conferring spatial and temporal regulation of the endogenous TRP-1 mRNA. Such regions, which are normally located immediately upstream of the transcriptional initiation site, can then be positioned immediately upstream of an exogenous gene, in this case a reporter gene, such as firefly luciferase, chloramphenicol acetyltransferase(CAT) or fluorescent proteins, such as green fluorescent protein derived from Aequorea victoria. These reporter proteins allow sensitive detection of changes in levels of transcription when monitored by appropriate instrumentation. Cell lines can be then created by stable transfection of these reporter constructs, and various molecules can be added to these cells to determine the degree to which the transcription of the reporter construct is affected.

Changes in levels of translation of the endogenous TRP-1 protein can be readily monitored by an assay which measures the amount of TRP-1 protein by reactivity to an antibody which is generated to recognize the endogenous TRP-1 protein or a sub-fragment thereof. Appropriate antibodies can be coupled to various enzymatic, fluorescent or calorimetric conjugates, and the increase in protein levels can be compared under various conditions.

The following Examples illustrate the present invention, without in any way limiting the scope thereof.

### EXAMPLES

Methods

Databases and Software

BLASTN searches are performed on release 16 (May 23, 2000) of the Celera Human Genome Database, which comprise 13451256896 base pairs in 22851604 unassembled sequences. For HMM searches, an amino acid database is generated by translating the genomic sequences in all 6 reading frames and retaining amino acid sequences of at least 50 residues. Prior to translation, repeat sequences and vector sequences are masked by Celera. The resulting ORF peptide database contains 34065236 total sequences.

EST databases used are dbEST, LIFESEQ™ GOLD database v5.0 (Incyte Pharmaceuticals), and the Novartis FGA cDNA library EST sequences. To confirm novelty of selected sequences, comparisons are performed against NT, the nonredundant set of nucleic acid sequences from GenBank, and NR, the nonredundant set of amino acid sequences translated from GenBank entries (Benson 2000). HTG draft sequences from the public human genome project are collected from GenBank.

BLAST searches are done with BLAST version 2.0.10 (Altschul 1997; Altschul 1990). Genscan v1.0 (Burge 1997) is used for genefinding. HMM construction and searching are done with HMMER 2.1.1 (see Eddy, SR, unpublished). GCG SeqLab is used for editing sequence alignments.

HMM Construction

To ensure a complete representation of the superfamily, an updated HMM representing the recognized cysteine rich domains of all known TNFRs is built. The peptide sequences of all human members are collected and used to query the NR database for homologs in other species. All sequences are split into pseudorepeats which are aligned based on the Pfam (Bateman 2000) HMM model PF00020 (December 1999 version) for the cysteine rich region. Sequences are filtered to a maximum of 80% identity and the alignment used for HMM construction.

Gene Structure and Localization

The genomic sequence for TRP-1 is built by alternately BLASTing the growing gene against human genomic sequence databases and assembling the new sequences into the contig. Public HTG sequences are split into component contigs prior to searching. Since the Celera genomic sequences are short, unassembled reads, the longer public HTG sequences are used preferentially in the assembly process. A genomic consensus is built from an alignment of the component sequences. Sequences included in the consensus are AL353136.4 and AC023560.2.

The exon structure of TRP-1 is computed by aligning the cDNA to the manually assembled genome sequence with sim4, November 1999 version (see Florea et al., Genome Res., Vol. 8, pp. 967-974 (1998)). The chromosomal location of the gene is determined by mapping the cDNA sequence to the Celera assembly (Aug. 15, 2000 Release).

DNA Sequencing

Clone 5091511 is obtained from Incyte in the cloning vector pINCY and sequenced by ACGT, Inc., Northbrook, Ill. Sequence ambiguities are resolved by comparison with available genomic sequence, emphasizing the high quality Celera reads.

RT-PCR

Expression analysis is performed with the primer combination, 5′-ggctgggtgagatgtgtgctctgcgctg-3′ (SEQ ID NO: 4) and 5′-gcattagaggtagctgtgcagttgaccttc-3′ (SEQ ID NO: 5), which amplifies a fragment of 340 base pairs. PCR conditions are: 1 cycle of 95° C.×2 minutes, and 35 cycles of 94° C.×30 seconds, 68° C.×1 minute, followed by an additional extension period of 10 minutes at 72° C. Human Marathon Ready cDNA (Clontech) is used as a source of tissue specific cDNA. PCR products are electrophoresed on a 3% agarose gel and visualized with ethidium bromide.

Northern Blot Analysis

Probes are generated by excising the insert of Incyte clone 5091511 from the cloning vector pINCY with restriction enzymes EcoR1 and Not1. The TRP-1 cDNA fragment is gel purified using Qiagen Gel Extraction Kit (Qiagen) and labelled using Rediprime II DNA Labeling System (Amersham) and α³²P dCTP (NEN). Hybridization of Human Poly A⁺ RNA Blots (Origene) and human total RNA blots (Clontech) with radiolabelled probes is performed for 18 hours at 60° C. in ExpressHyb (Clontech) supplemented with 100 μg/mL sonicated salmon sperm DNA. The membrane is then washed at high stringency and subjected to phosphorimager analysis (Molecular Dynamics).

FLAG-Tagged TRP-1 Expression in 293 Cells

The putative extracellular domain of TRP-1 is amplified by PCR with the oligonucleotide primers 5′-gacaagcttatggattgccaagaaaatgag-3′ (SEQ ID NO: 6) and 5′-ccgggatcctctatcaaagtgtggcctcctgaggg-3′ (SEQ ID NO: 7) using Incyte clone 5091511 as a template and the resulting amplicon is digested with the restriction enzymes HindIII and BamH1. The fragment is then ligated into the expression vector pFLAG-CMV1 that has been digested with HindIII and BamH1. The resulting ORF thus encodes a protein that possesses the pre-protrypsin secretion signal, followed by a signal peptidase cleavage site, and the mature protein consists of an amino-terminus FLAG-epitope followed by the extracellular domain of TRP-1. This construct is linearized with ScaI and introduced into HEK293 cells via calcium phosphate transfection with 1/10 concentration of an expression vector carrying the neomycin resistance gene, and stably transfected cell clones are selected in the presence of 500 μg/mL G418. The conditioned medium of cell clones is analyzed for the expression of secreted TRP-1 by Western blot analysis using anti-FLAG monoclonal antibody M2 (Sigma). Two clones displaying the highest level of TRP-1 are expanded and conditioned medium is collected and frozen.

### Example 1

### Identification of a Novel Tumor Necrosis Factor Receptor Related Protein-1 DNA Sequence Using Bioinformatics

TNFRSF members are defined by the presence of one or more copies of a cysteine-rich domain in their extracellular regions. To search for novel TNFRSF members, an updated Hidden Markov Model (HMM) is built from the cysteine-rich domains of all TNFRSF members in the GenPept database. This HMM is then used to query a database of translated ORFs derived from the Celera Human Genome database. Sequences of hits matching the HMM are collected as are the nucleotide sequences from which they were derived. Novelty of the hits is determined by BLAST searches of the NR and NT databases. Membership of any novel sequence in the TNFRSF is assessed by visual inspection of the BLAST and HMM search results.

One selected hit has a statistically significant match to the HMM (Evalue=5e⁻⁵) and is found to be novel by comparison to all nucleotide and protein sequences in GenBank and GenPept. This translated ORF retains all 6 cysteine residues of the cysteine-rich domain defining the TNFRSF as well as other conserved features of this domain, notably a Y conserved as either Y or F in almost all of the known TNFR superfamily members (TRP1_{—}1 in FIG. 2).

Further characterization of this novel putative protein, named TRP-1, requires prediction of its full-length sequence. This is done in a three step process. First, the complete gene is built by iteratively searching human genomic databases for sequences which could extend the gene in either direction and assembling the overlapping genomic fragments with the growing contig. Second, the cDNA sequence is predicted from the assembled genomic sequence using a combination of theoretical gene-finding methods, alignment with EST sequences, and homology to known protein sequences. Third, the cDNA sequence is translated in the longest reading frame.

In order to confirm the predicted cDNA sequence and determine whether TRP-1 is an expressed gene, the genomic sequence is searched against EST sequence databases. A template apparently derived from this gene is found in the LifeSeq database, suggesting that TRP-1 is indeed expressed. This template, 304945.1, is comprised of a single 5′ short read of a clone from a uterus library. The clone, 5091511, is obtained from Incyte and fully sequenced.

The TRP-1 protein sequence is of a size and structure consistent with TNFRSF membership. The full-length cDNA for TRP-1 is set forth in SEQ ID NO: 2 and the deduced amino acid sequence of TRP-1 (269 amino acids) is set forth in SEQ ID NO: 1. The genomic DNA sequence for TRP-1 is set forth in SEQ ID NO: 3.

The TRP-1 sequence shows significant similarity to and only to TNFR superfamily members. It has an N terminal region with similarity to other TNFRSF members, a single putative transmembrane region, and a divergent C terminal region. HMM searches against the PFAM database have identified 2 cysteine-rich domains in the N terminal region (FIG. 2). When compared to proteins of known function, TRP-1 is most similar to that of TNFRSF19 (TROY, TAJ) (aligned in FIG. 3).

### Example 2

### RT-PCR of TRP-1 from Various Tissues

To confirm the expression of TRP-1 and to study its tissue distribution, an RT-PCR experiment is carried out as described above in the Methods section. The gene is found to be expressed in a variety of tissues, with highest expression in uterus, prostate, testis and mammary gland. This is consistent with the Incyte clone, which is derived from a uterus library. PCR primers are chosen that span an approximately 11 kb intron in the genomic locus to rule out the possibility that the signal could be derived from genomic DNA contamination.

### Example 3

### Northern Blot Analysis of TRP-1 mRNA from Various Tissues

Northern blot experiments are carried out as described above in the Methods section to further characterize the expression pattern. An initial sample of various tissues reveals highest expression in the stomach, with weak expression in heart. A more detailed examination of digestive system tissues reveals apparently specific expression in the stomach. The slight difference in message size in heart and stomach suggests that, like other TNFRSF members, TRP-1 may be subjected to alternative splicing.

### Example 4

### Expression of FLAG-Tagged TRP-1 in 293 Cells

Having shown that TRP-1 is an expressed gene and some of the tissues in which it is both expressed and absent, the next step is to characterize its function. Since extracellular binding is the physiological trigger of downstream responses for other TNFRSF members, determination of the natural ligand or counter-receptor of TRP-1 will greatly facilitate function characterization. The ligand may be identified by receptor cloning if a soluble form of TRP-1 is available. To make such a reagent, constructs are designed that encode a protein encompassing the extracellular domain of TRP-1, amino acids 3-139 of SEQ ID NO: 1, with a FLAG epitope (DYKDDDK) at the amino terminus. The secretion signal of preprotrypsin is added to ensure proper secretion. This recombinant protein is efficiently secreted when transfected into HEK293 cells as determined by analysis of the conditioned medium by Western blot with the monoclonal antibody M2, which is highly specific for the FLAG epitope.

What is claimed is:

1. An isolated polypeptide comprising an amino acid sequence selected from the group consisting of:
(a) the amino acid sequence set forth in SEQ ID NO: 1; and
(b) amino acid residues 1-136 of SEQ ID NO: 1.

2. The isolated polypeptide of claim 1, wherein the polypeptide consists of the amino acid sequence set forth in SEQ ID NO: 1.

3. The isolated polypeptide of claim 1, comprising the amino acid sequence set forth in SEQ ID NO: 1.

4. The isolated polypeptide of claim 1, comprising amino acid residues 1-136 of SEQ ID NO: 1.

5. An isolated DNA comprising a nucleotide sequence selected from the group consisting of:
(a) a nucleotide sequence that encodes a protein comprising the amino acid sequence set forth in SEQ ID NO: 1;
(b) a nucleotide sequence encoding amino acid residues 1-136 of SEQ ID NO: 1;
(c) a nucleotide sequence as set forth in nucleotides 113-922 of SEQ ID NO: 2;
(d) a nucleotide sequence capable of hybridizing under high stringency conditions to a nucleotide sequence as set forth in nucleotides 113-922 of SEQ ID NO: 2;
(e) a nucleotide sequence of SEQ ID NO: 3; and
(f) a nucleotide sequence which is complementary to the nucleotide sequence of (a), (b), (c), (d) or (e).

6. The isolated DNA of claim 5, comprising a nucleotide sequence that encodes a protein comprising the amino acid sequence set forth in SEQ ID NO: 1.

7. The isolated DNA of claim 5, comprising a nucleotide sequence encoding amino acid residues 1-136 of SEQ ID NO: 1.

8. The isolated DNA of claim 5, comprising a nucleotide sequence as set forth in nucleotides 113-922 of SEQ ID NO: 2.

9. The isolated DNA of claim 5, comprising a nucleotide sequence capable of hybridizing under high stringency conditions to a nucleotide sequence as set forth in nucleotides 113-922 of SEQ ID NO: 2.

10. The isolated DNA of claim 5, comprising a nucleotide sequence set forth in SEQ ID NO: 3.

11. The isolated DNA of claim 5, comprising a nucleotide sequence which is complementary to the nucleotide sequence of (a), (b), (c), (d) or (e).

12. A vector comprising at least a fragment of the isolated DNA according to claim 5.

13. The vector according to claim 12, comprising transcriptional control sequences.

14. A host cell comprising the vector according to claim 12.

15. A method for the diagnosis of a disorder selected from the group consisting of an inflammatory disorder, an immune disorder, a neurodegenerative disorder, a cell proliferative disorder, a cell differentiation disorder, an apoptotic disorder, a gastrointestinal and reproductive tract disorder, bone disorder, blood disorder and viral disorder in a human, the method comprising detecting elevated or reduced transcription of mRNA transcribed from a natural endogeneous human gene encoding the polypeptide according to claim 3 in an appropriate tissue or cell from a human, wherein the elevated or reduced transcription is diagnostic of the human's suffering from the disorder.

16. The method of claim 15, wherein the natural endogeneous human gene comprises the nucleotide sequence set forth in SEQ ID NO: 3.

17. The method of claim 15, wherein the step of detecting elevated or reduced transcription of mRNA transcribed from the natural endogeneous human gene encoding the polypeptide comprises contacting a sample of the appropriate tissue or cell or contacting an isolated RNA or DNA molecule derived from the tissue or cell with an isolated nucleotide sequence of at least about 20 nucleotides in length that hybridizes under high stringency conditions with the natural endogeneous human gene encoding the polypeptide.

18. A method for the diagnosis of a disorder selected from the group consisting of an inflammatory disorder, an immune disorder, a neurodegenerative disorder, a cell proliferative disorder, a cell differentiation disorder, an apoptotic disorder, gastrointestinal and reproductive tract disorders, a bone disorder, a blood disorder and a viral disorder in a human, the method comprising detecting an amount of a polypeptide according to claim 1, or fragments thereof, in an appropriate tissue or cell from a human suffering from the disorder, wherein the presence of an elevated or reduced amount of the polypeptide or fragments thereof, relative to the amount of the polypeptide or fragments thereof in the respective tissue from a human not suffering from the disorder is diagnostic of the human's suffering from the disorder.

19. The method of claim 18, wherein the detecting step comprises contacting the appropriate tissue or cell with an antibody which specifically binds to a polypeptide that comprises the amino acid sequence set forth in SEQ ID NO: 1 or a fragment thereof and detecting specific binding of the antibody with a polypeptide in the appropriate tissue or cell, wherein detection of specific binding to a polypeptide indicates the presence of a polypeptide that comprises the amino acid sequence set forth in SEQ ID NO: 1 or a fragment thereof.

20. An antibody or a fragment thereof which specifically binds to a polypeptide according to claim 1 or a fragment of the polypeptide.

21. An antibody fragment according to claim 20, which is an Fab or F(ab′)₂ fragment.

22. An antibody according to claim 20, which is a polyclonal antibody.

23. An antibody according to claim 20, which is a monoclonal antibody.

24. A method for producing a polypeptide according to claim 1, which method comprises culturing a host cell having incorporated therein an expression vector comprising an exogenously-derived polynucleotide encoding the polypeptide under conditions sufficient for expression of the polypeptide in the host cell, thereby causing the production of the expressed polypeptide.

25. The method according to claim 24, the method further comprising recovering the polypeptide produced by the cell.

26. The method according to claim 24, wherein the exogenously-derived polynucleotide encodes a polypeptide consisting of an amino acid sequence set forth in SEQ ID NO: 1.

27. The method according to claim 24, wherein the exogenously-derived polynucleotide comprises the nucleotide sequence as set forth in nucleotides 113-922 of SEQ ID NO: 2.

28. The method according to claim 24, wherein the exogenously-derived polynucleotide consists of the nucleotide sequence as set forth in nucleotides 113-922 of SEQ ID NO: 2.

29. The method according to claim 24, wherein the exogenously-derived polynucleotide comprises the nucleotide sequence as set forth in SEQ ID NO: 3.

30. The method according to claim 24, wherein the exogenously-derived polynucleotide consists of the nucleotide sequence as set forth in SEQ ID NO: 3.

31. The isolated polypeptide of claim 1, wherein the polypeptide is pegylated.

32. A pharmaceutical composition comprising the polypeptide according to claim 1 and a pharmaceutically acceptable carrier.

33. The pharmaceutical composition according to claim 32, wherein the polypeptide is pegylated.

34. A method of suppressing inflammation in a subject comprising administering a therapeutically effective amount of the polypeptide of claim 4.

35. A method of identifying a molecule that binds to and/or modulates the activity of a polypeptide according to claim 1 comprising:
(a) contacting an isolated cell which expresses a heterologous nucleic acid encoding the polypeptide with the molecule; and
(b) determining binding and/or modulation of the activity of the polypeptide by the molecule, to identify molecules which bind with and/or modulate the activity of the polypeptide.

36. A method of identifying a molecule that binds to and/or modulates the transcription or translation of a heterologous nucleic acid encoding a polypeptide according to claim 5 comprising:
(a) contacting an isolated cell comprising the nucleic acid with the molecule; and
(b) determining binding of the nucleic acid and/or modulation of the transcription or translation of the nucleic acid by the molecule, to identify molecules which bind to and/or modulate the transcription or translation of the nucleic acid.


SEQUENCE LISTING (7 sequences)

SEQ ID NO: 1 - protein, 268 aa, Homo sapiens
MDCQENEYWDQWGRCVTCQRCGPGQELSKDCGYGEGGDAYCTACPPRRYKSSWGHHKCQSCITCAVINRVQKVNCTATSNAVCGDCLPRFYRKTRIGGLQDQECIPCTKQTPTSEVQCAFQLSLVEADAPTVPPQEATLVALVSSLLVVFTLAFLGLFFLYCKQFFNRHCQRGGLLQFEDKTAKEESLFPVPPSKETSAESQESFTMASCTSESHSHWVHSPIECTELDLQKFSSSASYTGAETLGGNTVESTGDRLELNVPFEVPSP

SEQ ID NO: 2 - DNA, 1156 bp, Homo sapiens
gggagggggctgggtgagatgtgtgctctgcgctgaggtggatttgtaccggagtcccatttgggagcaagagccatctactcgtccgttaccggccttcccaccatggattgccaagaaaatgagtactgggaccaatggggacggtgtgtcacctgccaacggtgtggtcctggacaggagctatccaaggattgtggttatggagagggtggagatgcctactgcacagcctgccctcctcgcaggtacaaaagcagctggggccaccacaaatgtcagagttgcatcacctgtgctgtcatcaatcgtgttcagaaggtcaactgcacagctacctctaatgctgtctgtggggactgtttgcccaggttctaccgaaagacacgcattggaggcctgcaggaccaagagtgcatcccgtgcacgaagcagacccccacctctgaggttcaatgtgccttccagttgagcttagtggaggcagatgcacccacagtgccccctcaggaggccacacttgttgcactggtgagcagcctgctagtggtgtttaccctggccttcctggggctcttcttcctctactgcaagcagttcttcaacagacattgccagcgtggaggtttgctgcagtttgaggctgataaaacagcaaaggaggaatctctcttccccgtgccacccagcaaggagaccagtgctgagtcccaagagtcctttaccatggcctcctgcacctcagagagccactcccactgggtccacagccccatcgaatgcacagagctggacctgcaaaagttttccagctctgcctcctatactggagctgagaccttggggggaaacacagtcgaaagcactggagacaggctggagctcaatgtgccctttgaagttcccagcccttaactctaatgaggtctcttgggcccctggcagccttgcccagttgttctctctggactctgttcctataccacaacagcagcaggggcctgaaatgtgatgtccacaagagctaataccctacagatggggcatatcctatcccatcccaccagaggattgattctccatttcacaaggactgatctggagcatttcttgcttccctgttgtagtctggggagccagattccacatgcatggg

SEQ ID NO: 3 - DNA, 18540 bp, Homo sapiens
ctttagccccttcattaaaattcctctttgtagaagacaacctttctgcatacttaactctgtcttattttgtatagctctttagaatggtttgatggaccttagctcctttaaccctcccaagagctatatacaataggtgaagaacattctatccctttttcttagattaagtaaccgaggccagaaaagataaagtgatccatcccagattgtagattgaaagggtaagagccaggcttggtgaacagccatcccacttcaatgctagctctactgctatatgcatctgtattccaagaactattatctgagtaatgtttccctatctgtgcacctttgtgcaccaaaatatgtctacattctgggttattctcccctcccattctgcctaagacagaagctatggcaggtattggtcctcagaggtactgaggccactgttggcaaagagaaacaatgtttcagctttttcagttataaaataaagatgacaatctctataatgcttacctcacatggatgttgtgtagatcaaataataatagatgtgaaagtactttctaaattgttatgatctgttcacacatgagggattgtgattattctttaccccctgggcagaagccaactccttgtcttattactctctttacaaattcagctcctggtatagccactgactgtgaatttgagcaaattgcttggtctctttgcctcagtttcttcatctgtaaaatgttgctaacaataattcttaatatgattgtcttgagaatggaatgagttactacatgcaaagcatttacaagaactcatggcacatggtcagagctacataagcattaactcttttgtgttggcttcctcgtccctactatctcagatctagagaatagattaaggggacccagaagccaagtggcatggaagccactacttatagtcccttcccactagcttgctagttcttgtgttctctacccacagccttcccaccatggattgccaagaaaatgagtactgggaccaatggggacggtgtgtcacctgccaacggtgtggtcctggacaggagctatccaaggtaagttatcctattcagtacacttatttctgttgcatcttaggaaccagcccggcaactgaaagaggccaagattaggggagtcagccctacttatgactgatccagttcagcgggtaaaacctgggatccacagagtatgcaatagcagtcatgattccagcatgaagtcagcctagcaggatcaaggtcaagggaagtttccatgagataacagagtatttcaagaatagatgtgtaatgcagagacccatgaggactgttcagcattagggtaatctgtcatctaaagcaaggcccaaccacatggtgttcaagagccagatatcaggtcttaggaatgagaaagagagaagagccaagaagaccctctgttctagagggtctgcgctactgaggaagctgtgcaatggagaatatagggatagggaaccacggagggtctaaaatcccaattaaagataaggatcagaataaaatgaccagcaaggcagagactttatgtaacttacttgtttcaagcctggatctgcttaattgttctttcctcaggtcccttgtaggtccctgaacttgggggtggtattaaattttggctgcagctggggctacagaggccagaagcaagcagctagcagatggtttctgcatattcacactctattttatgcaatttttgtttattcagcagttgcttattgaacacttgcagagtggcaggcatggttgtagacaccatggatatggcagaacaaacaaaaatatttgccttcatggaccttaccttctattattagtgaaaaaataataattgagacacatacagagataaatgaagaatctggatatgaagatatgagaagaatcaaattagggtgactgggtggctacttcagattatgaaacaaggaaaggtctccctaaggagacagcctctggggagaggatactagtgcagagtcccaaggtgaaaatgaactaggcattttgaaggaaaaggaggaaggtagtgcaatgggtgtatggtgaatgagagggagggtagtacacaaagaggtcagagagagaagtatagaccagaccagatagcagcttgaggtctatgagaaattttcatctttctctaagtgcattaagagctctttggagggttttgggttggaaagttataggaatctgtggctggaaaacaacataataattatctagcccactttattcctacctttgtattttaaaaaatgaagagaataaagcgcagggatatcaagagatacaagcactaattctaagtttttacacatggcattatgtcccacagtggggtacccacatggttgataaaggatcccagtaccaaggaaagatcgccagaagtaaggtcatgctggacattaaaatagatattaaagcttatatatgtgtaatacatataagctttagagccagatacacatgggcatgagtcccatctttgctctgggcaaattatttcacctcagaatcaattaggatgtagtaagttgaaagtaacataaaatttaaccatgacatgaaccataagtatatttatttatgtaatgaagtctggagacaggcaacctttttgcttgttgggcagatcagcaatgttatcaaggactcagcctcttttcatccttcatattggacatcttgctgttttgttctcattgtggcctcatgattgcaaaactcaaagcatcatatctccatacaatagtcaaagtcaggaatcaggggcaaggtcaaaatagcttgccttttgtagggaagaagtcttttcccagaagctgtccaacagatttttccttatatcttatggtccagaactgggtgatgtagctatctttagctggaatagagactgggaaataagtatctggccaatgagaatgggattgtgtgattaagaccaatcaagaatccgttctggaggctagtcttcatgccacctagaatgaaatcagtgtttttatagtaaggaagaagaggggcaattgtttgaataagcagctagaagcatctgatagccttttttaaagtaacttttatttacttttaaattttactcatgaattataaacatcactgtaaataaatcattgcagaaaattaacaccatttcctcaggacatttttcttgatgtataattgccaagctaaataatgcgtacgattttaagactcttgaaatatatcagcagataacacttctgaattttgtttatactaatttctattaccattacatttattttcaacagtgcatagaattcctgttgtggccctctgttcggggtccctgacttcccacaacaaattcccatgtcccagaagcctaaacgcaaatgaataatagatattttcatgatctattaattttatgccttccttttaacagtaattactaatttatgaggactttatgagaattttggttttactttttatctatttgataggggaaatgtaccaactttactgtgtttatttgcccatttgcctttccaattttgtgatctatttctgattgtttctctttctattctttctattcattatgtattgttctttgtctactcacataaggaacctaagtagacagacatatagtagataacagagtattttaaaaatcgtttaaaatgtaagtatatttgtatttaaaaaccagaaatgcttttaaattttgtttcattttagtatcatctgttgatttgtaaatatattttccctttgatgtattaatttgatgacatgtattaataaattcctaaaatttaaatatcctgcattcccagtataaaccattcttgattatagttttaaggtaaatacccttttatgttgtataagttagtattttaactaaaacaaataattattattgtaacactatcagcatcaaaattttggatcagcattgtgctaccatggcaaaatcaagaatatcaatgttttctggctataaaagtggttaatagcaaaagaaattttgcaccttaatgatttgaaagaacttcctttgcctcctaattatgaaggagaattctcaataaatttttaatgataatttgctccttaatttattaaaaattattcatttatttaatttttattgagtacaatttgatagtctttggttttatttcactatgccaatccctgtcttttaattggcttacttagaacatttatgtttaaataatctttgatattttaagatttaaacctaacattttattctttttgtgtttgttttctctgtttcttttgtccctctatttgtcttttcttgccttcctgtgggttccatgaacattttatttaggattccatcatgatttatttgtagtgtttttgagtgtgtctctttgtatcatttttttagaggtggttctgagtattacatgaaaaactaacacgttactttttcaatgtcagctggtatcaacattttgccatttcaagtgaagtattgaaatatctgttccatttagattcctttactttccccacttttaaatatcattgtcacgaatatcagatataatttttgttaaatttgttttagtcatctactatgatttataaaatgtatgaagagaaagatagtacattttatacaaccatattttttctttcccagtgttccttccttcctgatgctccaagatttcttcttttatcacttccactctttcttaaatcatttcatttgtgttttatgaactctctttagaattatttaagggtaggcctactagcaactcattctgttagttttcctttgcctgagaatgtttttatgtcccttcatttctggggatactttcactggatataaatttgcagttgacagtttttttatttcagtacttgaaaaatgtaatacttccttcttggcttccatggcttctgacagaaacccactgtcgtatcatttgaatgggtgtttttctataggtaatgccatttattcttggctattttcaatatattttttttagttttcagaagttttattatgatgtgtattggtgtgcattactttggatttttttctgtttgggatctgttcatcctctttaacttgtaggtcttttaccaaatttgtgaagttttcttccaatttgtttgaaaatatttttagataaacatttaaaaaattttcttttcataagattctgacaatatgatttttctatattttgttatagtctcataggttcttgaggctttgttcattttattttgtcattttctccctgttgttcatattgcataaattctgttgttctgtcctcaagttcactgattctatcatctgtctcttctctactactaagcccatccagtgaatttttaaaatttctgtcattttatttttaataattaccatctgtttatttaaaaaaaacttctgtttctttgatgagattttctatttttaagagaattttaaattgttttttaaaacatttttattatgggtgctttagaatccttgttcaatcattccaacatctgctttatctaagagttgctgtcagttaattgacttttctcattcatgttgtgattttccttattcttattatcatgggtggtttttcgttgtgttctggacattttgtcaattatgtcaggagactcagggtcctatttaaacttttactttagcaggcagtcatctgtttatgtttagcatacaggttttggcctacgtttgtgagctgcagttctaaaggctatttaatttccagaacctttgtagtgtgatttttggtctgcggggtttatcttgtgctgcctcaggtatagacgatttcctgggccaggtcacttggtccatttaggtcggcataaagaagtgtctgagttgtagagtcaaagaacatttcccatgctcgaccacttgttattagtaggataccacttgccagtataccccatccacctgttcctatgtgggggaaaataatctcagacccacaggacaaagaagcatttgagctggttacttatggtggaatctcaagtgccccctgcagcctattgtctctagttgtgtgtggggaatcgcaggcccatggggaagaggcttctcaggccaacaatttgtgcctcaattttgcttatttgtgaaacatggattatattgcacccttataaggttttatgatgaataaacaagtatataaattcatgagtgtgtggatttttgtctgattgattcaattatttagtcacaatattcagaatagcatcctcatgcattgtagttgcttcaaaaatgttttctgagttaaccaataaaatgaaagggagtgctaaccaattaatcaatcaatcaatcactcaacttattggttaactttcaataaatgctagcttgcttttgtcattcccttctctagcaagtgagtaagtgaggagtcaggcacatatcttctgattccagtgctatagatagaataaaaacttgctttccagaagaacaatacctaatgttcatctgttcattgaaggattactatgttctaggcatggtgacaagcactacacacaacccacacacagagctatatatgtatctgtgtgtgtgtgtgtgtgtgtgtgtgtgtgtgtgtgtgtgtgtttgtgtatctggttaagagacatcctttatctaaataagagctgtttgtgtttctttttctgtgaatatatgtttagtttctcataaggtggttgtcgttaatttatactagttctgtgttatcggaatataaatctttagtctgtgatatggtgtacatctttttactagttcattatttgtcttctaattttgcttatgaatcttttaccctgaaactttttaaaatttttatgtaattatatctatctttcctttactgcttctggacttcgtgttgcagttagagaaaatttctccactcccagattaaagaggaatttatttatattttgttttggtactttttggttttaatttttatctataaatttctgatctattttggtgtatagtgtgaggaatgtatcaaattttatcatttttcacattgtcattcggttatccaacataacctatttaaaaagccagctattctccattgctttgggatattccttttgttcatttggtcagctcatgtaatcttaacaaatctataagttggatagcattatttccaattggattgacaagaaaattcaggcccaatgacttgcccaaggtcacatcgctggtcagtagtaaaagtgggagttaaagataggtcttcattaccttggatcagggattttcatcactacattatatttcctaataatttgacccccagctcactgttttaagcagatatggaagctatcaccattccagtggtgcacataaggtagcagagattccaagaaaccctttaacttttcaagaaacttagagcatgagtaaaatgactactgcccatgtcagatcccattggtagatgggatcattgtgagtgaggctgcaaggcattgagtaaagcctgagtgggcaaacatggggcagaatggaaggagtatttcgagcctgcctgatgctcactttttccaactcctgggaaagtataacttaaatgttagttcaggtaatattaactgctgtaacaaatactaaagtttggtgacataagaggttaagggtttattcctgaatgtgactccaatccatgttggggagtaggaagattcttctcagccattcaggaagccaggcttatgggagctctgtgttttttaagcagccttattaaaatacagttcacataccagtcaattcacccatttaggtttacaattcatggtttttggtatactgcattatgatttttaagattgtggtaaaatatgtaatctcaaatttgctgtattagacattgtaagtgtataattcagcagcattaatcacattcacaatgttatgcaaccgtcacccctttatttctaagactttttattaccccaaatacagtctctgtatgccttaagcaataactcccaatttccccctccccaaagtgcctgatagcctctactctctgtctctacaaattttgctattctagatatttcatgtaagtggaatcatattttttcatgcagtcacctttaccagagatatttactctttgcatggctttgggttactctccattgtctgttcatttcagcctgaaggactctcttcttgtagtcaggtcaaatgatatcaatcagctgttgtttgtctagggatgttttaattattcctttatttttgaagaacttttttctgtatatagagttcttggttaacagtttatttgtttaggcacttatatgtcatctcactgactttaagtctctgcactttgtgatgagaaatatgctttgaatcttatcgagaatacctttacttgacaagtaatttcactcttgattttcaatattttctctttgttttcagctataaacattttttgatagtgtatctcagcataggtctctttatgtttactctgcttggagtttcttgagcatcttggatgggttaatttatgtctttcatcagatttgagaagttttaagccattatttcttcaaatattcattctcgtcccccatctccctgttttttctgtgaatcccataatgcatatattggtcacctgcatgatgttccacaagttccttatgttctgtttactttcttctcctcagacttgataatttaaattgtcctacattcaggtttgctcattctttcttttacctactcaaattctctagtgaagttttcatttcatttatcttactttttagctccagtattcatgtttggttccattttacaatttttcttaaccaatattctcactttgtttatacatttgtttcctgatttcttttagttctttgtccatgtgtgtctttgagcaaatttaagacagttcttttagtctttgtctactaagtgcaatgtctgggcttcctcaagaatagtctcaattctttctttttcttgttcctttgaatgggccacacttgcctgtttctttttacactgtgtgatttatttttttgttgttaaaaactgggcagttttgtattgtaatgaagtaactctggtagtcagatttcccctcttcctgttttacttttggttttggttttgtttgtttgttttctagatttgtgttgttgaaggctatagtagtccatctgttgttgttattattatcattattgttattattattattttgtttgggactgggtattgctctgtcaccccaactggagtgtagtgtcatggttattgctgacttcagcctccacttcccgggctctagcgatcctcccatctcagccacctgagtagctgggaccacaggtgcatgccaccattcccaactaatttttgtatttttggcaacaaggctttgccgtgttgcctaggctagcactccatttgtttaggtgtttttccaaactatttttgtagagactattttttgtcatgtatcgccattgaaacctatgttaatttagctcatgttcagctaatgtttggacagcaatttcattgaatgctaaaagttaaaaaatgacaacaacaaccaaaagaaaaaatagtcacacattactctcacatttttagattggctctgtgctggggaactacttaaacagttagccagatgtgcacttagcttagggatcagcctgacatgtaagctttaggtctccactagtattttctgagcatgtgtcttgtatgcacacacctttctaaattcctccatgtggatacttttgaatgtacttatttctaaaggaacctcccccagcttttgctcccagattcagacagtctgttttatgtcttaatccataatcattgccctaggtattgtggtttgttagtttgccttgcaacattttgaaacagtgcccatcacttttccaatctgcattctcaatcgagtggaacatacataaataccttgtatcagtccttcaaatagccctcaaataggttaggacagacatacacagtaatttgtcaataagttttgcttagctcactctggaatcaggtactgaggtctcacactaggaatacaggctgaggctactttaagacttccactactttaggactgccactgctctggggtgagggatgtaacaagtacaagtaaaaatagcagaaaactttcctattattttcaagtgacctttttcttgatttaacatttacatggttgatgtaagcctttgaatgttttccagagttctgacaacattgattatgacagttcctgcttgtttttctatgtttctgtaggggcataagagcttgaagctgcctactcctccattttatctggcttccgctatcattcttaatatatttattccaaggtttccaggggtgtcagcatgcactggaaaatgaatgtatgaagaaaatttaagtatcacacaggacatgtttttgagtcaagcctgggaatggcaaacaatattagtatccatatttcattggctagaaatgatgcacatgcccccacttcattgcagtggggttaaagaattttggctaatggtgttcccaggagaaataaagaataggttaagtgaacaaccagccagcttctgctacaactagcatacaattaatatgctttcacaactagaattgtttctttgagaaagagctgtgactggtatcaggcaggagccttgttcctgttgaagttaggctgtcagatctaagaatctctagagagcatttatttcaatttcctcagtgttgggacttctgatgcccagaaactgggggtcggggggaaggaatatggccaaagttagtaccagagctgagacaagagccagctagagtgtttgcctctcatctataccattggcattcaaacattttttaccatgacctagagtaagaaataactttttatcacaacccaatatatactttcatgcattttatctgtaaaacttttataaaacagtacttatgtgggtagtgcttatgactatttgtcaaatatttcagctccccacattctgtcacattgtagaattgcattttcttgtacctgttgtagttattaatgatcatgtgatgtgtatggccaaggacatgtgagtaaagcaatatgggtcatttcttggggaaaactttaagagcttgtacgcagttcacaatgtttcaatcataaaagcagaaaaataaagcttctgtaaccccgggtaccccattgaggatgatgtacagcagagtcctcaggaaccccctggtaggcaaatatgatgagtgaggaaataaacttttattcttttaagacactgagatttagggtttgacttttgccatactgcagcatagcctatgctgactgatacaaatacttagctttacttcattccatgtactcttatatttgatactattattctatttcattctattttatctttcaatgctggctggaagctagtgaattggttttatgacttagtaatggggttgcaagttgcaaaatactaccctgtacagtccacttgtcccacaggtgatcccatctccaagctcctataaagcaatattaacaacccccatctgtctacaggattgtggttatggagagggtggagatgcctactgcacagcctgccctcctcgcaggtacaaaagcagctggggccaccacagatgtcagagttgcatcacctgtgctgtcatcaatcgtgttcagaaggtcaactgcacagctacctctaatgctgtctgtggggactgtttgcccaggtgagcttgctttatgagaccaagcttgtctgggcttttctagctgttggaggtttctcccatcctcacctctcagaacttagtcgaaatattcttcttataaacataatattcagtttaagctcccagaagagagaagtggtgtcttcccaagatacctctataaagtgcttagcacattgtcagtcacagatgagtacttgaccgcttttccatcatcactctcagctgaagaccaaagttcaaggtaaatctcactgtagacttaaaagaagttatgatgtacttggtatcaaagcagtgcacccctctcttgtggaattaggtctccttttggctacatggtgtcatgaatgggggaagaagtagagtgtctgggtctctgagtcagaaaaagacacagaggcagataactcagaaagaggggtgggcacttactttcaggttggcaagattcttaggagctgctttcccaggaagaactggtccaagtgcattgcttgtttgatcaccctgccattcctaattgctctgaacacaggttctaccgaaagacacgcattggaggcctgcaggaccaagagtgcatcccgtgcacgaagcagacccccacctctgaggttcaatgtgtgtacccagttctcttcttccctttctcctcatcccattgggtggcagcaagatagggtagtaaagactacccatatcccctcataccttttcatgtagagtagagtggcaagggaatcagattagaagtctggttttacaacatattatctttgcaactctatgcaaatcattcaatctcattgaacctcatttcctacttctgaaaaaagggtgagtcatgataatcattttttcttcataccttgtgtgatgggtatctaggtctaatgagaaaatggatgttaggaagcacttcacaaattataatatttgtaaacctatgagatattattattatatgaagtatataggagcccctagggctgaggacttgtaagaggctgaatgaagaactagggtccaatctatgcctataccactatccatttatctaatcgtctatcttttttgtctaccatctatttacttacttcctattttaaaattattgacatattttttaaatcatgaaagttttcaaacatgtagaaatatttttaagtgcagaaaaatataaaattaaaatgcaatcacccagcatctatccaaagttaataaatcatagcattttgcatatttgcttcagatgttttaaaagaataaagattccacagatataatggaattctcttctagattctctcagagtcctattacctccctacctcctcagatacaaccactatcctgaaattgtatgtattctgttggtaggctatgctaaagtaacaaacaaactgcaaaatctcagtgactttatacaataaaagtttattttttttttactatgcaaaggccttcctacatcatttgactgtgcattctagaccatatgtcttccaggtcaatgacacaggggaagagatggaggagacatactggttcttaactgcctcagtctggaagtgacacatgttatttcaactcacagttcattggccagaagtactctgatggccttactgcaagggtgtaagggaaatacatgggagtatgtggataactggtgagcactatacctctgccatactctccactaatgttttaatacttgggttcttttgtgatatgtgtgcatattcataaccaatatatgctattgttatgtattgtataatgttttatagaaatgatatcaagctatatgttctgtttctccaacttcctttttccccctctagattatttatattgatccataaaggcctttaagtgctgtataataatgtaagattttaggattatatcatgatttgtttatcttgtgctctactgatgaattttttagcttgtttccattgatctggattatataattagatgtggcacagaaagatcacaggatatgtgcatcttcgattaattgtactaatttctacataccagcactatatacaaattattttgccttgttctttataattttgcctgctctagaaagaagtaaaaccagcatagccaccctaattggggaaggccctagggagaagggtgacagcctgaggaagtccaggtccagggtcagggtgactgtttactgcccatgctaactgacctcccccatttttttgtctcacaggtgccttccagttgagcttagtggaggcagatacacccacagtgccccctcactaggccacacttgttgcactggtgagcagcctgctagtggtgtttaccctggccttcctggggctcttcttcctctactgcaagcagttcttcaacagacattgccagcgtggtaagggtggctgtttcatgtttcacatgaatcaggaacaaggctcttattggcagaagagagggatgttctggggttgagagacaatgggccttttctaccatgcttgaaaaccccatgaatagtcttctgttggcctggggaactatctggggctgcaatggaaagcatgggacttaagagattactgaagccaaccctgagagtttagaagacctggttcattatgtttcaggggattttaaaggaatgtggcacggtgggaaacccctcaggaaaggaatcgggagacctaggttctatgtctacctctaaatcaccgtgtaaccttagacaagtcatctagcctcttcagacctctttcttcctaatctttaagataagagataggattggatttgttgatttctgggttcttttccaattctttatatctatactagtaggtataaattctagagcatgtcatgaattgaggaaggcttggctatatctgagatacgactgtaataaaagtggttgaggtctaaggccactgctatcagtataataacttcacaaatggggataactgaaagccctatgtccagagtagttggcaaacctggactttctccctcagataatactgatgtcaacaggtctagcccagtccatatcagacagccatgcaaaattccagatggaaaatcatatttaaagtcacagtaacattgtgatctctattaagcacaacttcagctactagacaagaatgtgggacagaaaagccagctcactgaggacaaagatgggggaggtgggaatgtggctgatcaggaccaggcaaagagttagaactccagccaagagtaccaaggttcagtgcagcttcattgccattaagggaactgagagttaggcagggacactgaaacaggaactcagatcagagaatgaagttgatttataagggggaagataaagtttactagataaaagagtgctcaggactccttttcagcccactggatactgtgtgcaaagctgcagtatttgtgtgggaagatcctgctaatgtaaatttgccttgggtcagtttggctctggggcctagcagatgatcttgaaaagcccaagaggtatgtttggttgtgccctggcttaattggagatgaatatagcatttcatgggctgcaggatacacggggaacactgttgttcctttatgatttttctcaaagtctacttcttgctagctcctgggacactgtgactcaaggtagaccttcctgtctctgactttggaggccagtgtcccagataagagttgttgcttaggccctgggtgcatggacatgaaacatgagcaatgaagcatttggactccagggtccagaagatgaaaatgagaaaatatcagagagctgcctcagaagaagagagatgggtaacccttttctgctctccttctatcctgaagataataatgtggtaacctgcttggaatatgagggcaggcagtttcctcactgtttttataagggctaggaaggctagacattttcttactttggaaaactgtttttcaggagtcactgacttacacacatactggggtgagatgtatgtgcgaattaaatccagtgtaaggttgcaagagagaaaagtgcaactgtggcaaactacagagcacatcccttatctaaaggggaatcccactacttggatccagctgtttgtaaccatatggaaatatttaatgttgcaagatcttctacattttcaagagaattcagaaatctggattttttttaatgaagttgtgttaaccatagtcaatgaatttgaataaagagcaaaatcacaaccaaagcaaagaggacaaacacaaatcccataggttctcagtttgtgacctgatgttgaaccagatccatgcatgaagggggtattacttttctatcatggccataacaaattgccacacatttagtagcttattatcacagtttccatagctcagaagttcagtaggctcaattttgtctctgcttagagtctcacaatgccaaaattaagatattggcagggctgtgtttctttctggagtctctgggagagaatctgcttgatcatgcacaaaagtggcagaattcaggtctatgcagttaaaggacttaggtcccatttccttgctggctgtaagctgaaggtcattcctagcctctaggaccacttacattctttggctcatggcccccttctattttcaaaactagcaatgatgagttgagttcttctcatgccacatctctctgatactctctgtctcctctgccttcctcttctgcttttagggacacaggattacattggacccatagagtaatccaggattctctccttattttgaaatccataactttaattccatctgcagagtctagtttgccatgtaacttaatgtacatagacataagaccaggccatgaagattatggcaagggagatggtatcaaaatcctgtttcccacaaaggtttctttttctctagagaaattgattattttctctgatccagtaccagctagcctcaatctgatacctgaatgtaagtatgtttctcaactgtgtcacctcagcctggtgctaggtgctctagaccccagggagaggaaggaaagaatcagctcccagacctttacctgtcttgtactcagtacccagtgcccagctgagaagagcccctgcttttgggtaacccagtggctttctgcttagagctgcaggaggtttgctgcagtttgaggctgataaaacagcaaaggaggaatctctcttccccgtgccacccagcaaggagaccagtgctgagtcccaagtgagtgagaacatctttcagacccagccacttaaccatatcctagaggacgactgcagctcgaatagtggcttccccacacaggagtcctttaccatggcctcctgcacctcagagagccactcccactgggtccacagccccatcgaatgcacagagctggacctgcaaaagttttccagctctgcctcctatactggagctgagaccttggggggaaacacagtcgaaagcactggagacaggctggagctcaatgtgccctttgaagttcccagcccttaactctaatgaggtgagctggaaaggtggatcagaaaaaaacaaaaaaagcaaaacagaaattgaaggactaacctcaaggaattctcttttggaatagtattattcactaggagtggaggaacagcagcaagctaggaacactttgggtggaggggcaacagtggtgatgctaaacttttaagccagacagatatcacacttctgggtctacaattgtcagggaactggcaaggttgtcatgataccagtctctccattctggtctttcgtaagctctctcagggccccactatctaatggtcatattcaggtatctccctttatgtgtgcaagcagcatagcacagtagaaaaagatatacaagccttgtcatctggaaaccctacatttcaaatcaaccttcattccctaataactatacaaaccttgtgtgtatgtcacttcattttacattttacagaggcaggaactgagacacagagagggaaagttagtttctcaaagtcacagggcaaattagtgggctagaactgtgcaagaacccaggtttgggggctacattttagagttggttcaactccaccattgcaaccatttgatagccaatgttactttctcaaagcttatgtagcatgaggtaggagagtaggtagattcccagttttcaactaaccaaatatcttgcagtgcccacttccgtatttcttcagttaatacattttcagttctcccaatgacttccctgctccctgcatccaacctaagaaaacgtgaacaaatgtatatttgaaagtcataactgatatcaaatttgaaagttataactgatgtcaaatcaatcaacttaggtaccttcaaggttatctagtccagttcttcttccaaggtttgggtccattttataatatccccactaagagtttgtgtagtccttgctcacatactttaggtggcagagggctgactacttctcaatgtatattacagtatacacaattgt

SEQ ID NO: 4 - DNA, 28 bp, Artificial Sequence
  primer
ggctgggtgagatgtgtgctctgcgctg

SEQ ID NO: 5 - DNA, 30 bp, Artificial Sequence
  primer
gcattagaggtagctgtgcagttgaccttc

SEQ ID NO: 6 - DNA, 30 bp, Artificial Sequence
  primer
gacaagcttatggattgccaagaaaatgag

SEQ ID NO: 7 - DNA, 35 bp, Artificial Sequence
  primer
ccgggatcctctatcaaagtgtggcctcctgaggg